SEQUENCE LISTING (59 sequences)

SEQ ID NO: 1 - protein, 37 aa, Artificial Sequence
  rat amylin
KCNTATCATQRLANFLVRSSNNLGPVLPPTNVGSNTY

SEQ ID NO: 2 - protein, 37 aa, Artificial Sequence [sequence omitted]

SEQ ID NO: 3 - protein, 37 aa, Artificial Sequence
  pramlintide
KCNTATCATQRLANFLVHSSNNFGPILPPTNVGSNTY

SEQ ID NO: 4 - protein, 185 aa, Artificial Sequence
  Adrenomedullin (ADM) preprohormone
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 5 - protein, 37 aa, Artificial Sequence
  Adrenomedullin (ADM)
YRQSMNNFQGLRSFGCRFGTCTVQKLAHQIYQFTDKD

SEQ ID NO: 6 - protein, 32 aa, Artificial Sequence
  Salmon CT(1-32)
CSNLSTCVLGKLSQELHKLQTYPRTNTGSGTP

SEQ ID NO: 7 - protein, 47 aa, Artificial Sequence
  Intermedin
TQAQLLRVGCVLGTCQVQNLSHRLWQLMGPAGRQDSAPVDPSSPHSY

SEQ ID NO: 8 - protein, 115 aa, Artificial Sequence
  Preprocholecystokinin
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 9 - protein, 167 aa, Artificial Sequence
  Human leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 10 - protein, 147 aa, Artificial Sequence
  Metreleptin
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 11 - protein, 36 aa, Artificial Sequence
  Pancreatic polypeptide
APLEPVYPGDNATPEQMAQYAADLRRYINMLTRPRY

SEQ ID NO: 12 - protein, 36 aa, Artificial Sequence
  PYY
YPIKPEAPGEDASPEELNRYYASLRHYLNLVTRQRY

SEQ ID NO: 13 - protein, 36 aa, Artificial Sequence
  NPY
YPSKPDNPGEDAPAEDMARYYSALRHYINLITRQRY

SEQ ID NO: 14 - protein, 30 aa, Artificial Sequence
  GLP-1(7-37)
HAEGTFTSDVSSYLEGQAAKEFIAWLVKGR

SEQ ID NO: 15 - protein, 31 aa, Artificial Sequence
  GLP-1(7-37) analog
HXEGTFTSDVSSYLEXXAAKEFIXWLXXGXX

SEQ ID NO: 16 - protein, 230 aa, Artificial Sequence
  Fc portion of an immunoglobulin
AESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 17 - protein, 275 aa, Artificial Sequence
  GLP-1(7-37) analog covalently linked to the Fc portion of an immunoglobulin
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 33 aa, Artificial Sequence
  GLP-2
HADGSFSDEMNTILDNLAARDFINWLIQTKITD

SEQ ID NO: 19 - protein, 37 aa, Artificial Sequence
  OXM (also known as “glucagon-37”)
HSQGTFTSDYSKYLDSRRAQDFVQWLMNTKRNKNNIA

SEQ ID NO: 20 - protein, 151 aa, Artificial Sequence
  human natriuretic peptides A preprotein
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 21 - protein, 28 aa, Artificial Sequence
  Human ANP
SLRRSSCFGGRMDRIGAQSGLGCNSFRY

SEQ ID NO: 22 - protein, 121 aa, Artificial Sequence
  Rat BNP
MDLQKVLPQMILLLLFLNLSPLGGHSHPLGSPSQSPEQSTMQKLLELIREKSEEMAQRQLSKDQGPTKELLKRVLRSQDSAFRIQERLRNSKMAHSSSCFGQKIDRIGAVSRLGCDGLRLF

SEQ ID NO: 23 - protein, 32 aa, Artificial Sequence
  Rat BNP
NSKMAHSSSCFGQKIDRIGAVSRLGCDGLRLF

SEQ ID NO: 24 - protein, 126 aa, Artificial Sequence
  porcine C-type natriuretic peptide precursor
MHLSQLLACALLLTLLSLRPSEAKPGAPPKVPRTPPGEEVAEPQAAGGGQKKGDKTPGGGGANLKGDRSRLLRDLRVDTKSRAAWARLLHEHPNARKYKGGNKKGLSKGCFGLKLDRIGSMSGLGC

SEQ ID NO: 25 - protein, 22 aa, Artificial Sequence
  porcine CNP
GLSKGCFGLKLDRIGSMSGLGC

SEQ ID NO: 26 - protein, 40 aa, Artificial Sequence
  human urocortin 1
DNPSLSIDLTFHLLRTLLELARTQSQRERAEQNRIIFDSV

SEQ ID NO: 27 - protein, 41 aa, Artificial Sequence
  Human Ucn-2
IVLSLDVPIGLLQILLEQARARAAREQATTNARILARVGHC

SEQ ID NO: 28 - protein, 38 aa, Artificial Sequence
  Human Ucn-3
FTLSLDVPTNIMNLLFNIAKAKNLRAQAAANAHLMAQI

SEQ ID NO: 29 - protein, 25 aa, Artificial Sequence
  neuromedin-U25
FRVDEEFQSPFASQSRGYFLFRPRN

SEQ ID NO: 30 - protein, 25 aa, Artificial Sequence
  Pig U25
FKVDEEFQGPIVSQNRRYFLFRPRN

SEQ ID NO: 31 - protein, 38 aa, Artificial Sequence
  neuromedin U family truncated or splice variant
FLFHYSKTQKLGKSNVVEELQSPFASQSRGYFLFRPRN

SEQ ID NO: 32 - protein, 33 aa, Artificial Sequence
  human neuromedin S
ILQRGSGTAAVDFTKKDHTATWGRPFFLFRPRN

SEQ ID NO: 33 - protein, 39 aa, Artificial Sequence
  Exendin-3
HSDGTFTSDLSKQMEEEAVRLFIEWLKNGGPSSGAPPPS

SEQ ID NO: 34 - protein, 39 aa, Artificial Sequence
  Exendin-4
HGEGTFTSDLSKQMEEEAVRLFIEWLKNGGPSSGAPPPS

SEQ ID NO: 35 - protein, 39 aa, Artificial Sequence
  [14Leu]exendin-4
HGEGTFTSDLSKQLEEEAVRLFIEWLKNGGPSSGAPPPS

SEQ ID NO: 36 - protein, 37 aa, Artificial Sequence
  exendin-4 peptide analog
HGEGTFTSDLSKQLEEEAVRLFIEWLKQGGPSKEIIS

SEQ ID NO: 37 - protein, 30 aa, Artificial Sequence; identical to SEQ ID NO: 14
  GLP 1(7 36)NH2

SEQ ID NO: 38 - protein, 30 aa, Artificial Sequence
  exendin-4(1-30)
HGEGTFTSDLSKQMEEEAVRLFIEWLKNGG

SEQ ID NO: 39 - protein, 39 aa, Artificial Sequence
  Exendin analog agonist
XXXGTXXXXXSKQXEEEAVRLXXXXLKNGGXSSGAXXXX

SEQ ID NO: 40 - protein, 28 aa, Artificial Sequence
  Exendin analog agonist
XXXGXXXXXXXXXXXXXAXXXXXXXXXX

SEQ ID NO: 41 - protein, 44 aa, Artificial Sequence
  Exendin analog
HGEGTFTSDLSKQMEEEAVRLFIEWLKNGGPSSGAPPSKKKKKK

SEQ ID NO: 42 - protein, 50 aa, Artificial Sequence
  Exendin analog
KKKKKKHGEGTFTSDLSKQMEEEAVRLFIEWLKNGGPSSGAPPSKKKKKK

SEQ ID NO: 43 - protein, 38 aa, Artificial Sequence
  Exendin analog
HGEGTFTSDLSKQMEEEAVRLFIEWLWLKNGGPSSGAS

SEQ ID NO: 44 - protein, 42 aa, Artificial Sequence
  Exendin analog
KKKKKKHGEGTFTSDLSKQMEEEAVRLFIEWLKNGGPSSGAS

SEQ ID NO: 45 - protein, 42 aa, Artificial Sequence
  Exendin analog
NEEEEEHGEGTFTSDLSKQMEEEAVRLFIEWLKNGGPSSGAS

SEQ ID NO: 46 - protein, 42 aa, Artificial Sequence
  Exendin analog
HGEGTFTSDLSKQMEEEAVRLFIEWLKNGGPSSGASKKKKKK

SEQ ID NO: 47 - protein, 48 aa, Artificial Sequence
  Exendin analog
KKKKKKHGEGTFTSDLSKQMEEEAVRLFIEWLKNGGPSSGASKKKKKK

SEQ ID NO: 48 - protein, 48 aa, Artificial Sequence
  Exendin analog
DEEEEEHGEGTFTSDLSKQMEEEAVRLFIEWLKNGGPSSGASKKKKKK

SEQ ID NO: 49 - protein, 74 aa, Artificial Sequence
  Exendin analog
HGEGTFTSDLSKQLEEEAVRLFIEWLKNGGPSSGAPPPSGGGKCNTATCVLGRLSQELHRLQTYPRTNTGSNTY

SEQ ID NO: 50 - protein, 74 aa, Artificial Sequence; identical to SEQ ID NO: 49
  Exendin analog

SEQ ID NO: 51 - protein, 72 aa, Artificial Sequence
  Exendin analog
HGEGTFTSDLSKQLEEEAVRLFIEWLKQGGPSKEIISGGGKCNTATCVLGRLSQELHRLQTYPRTNTGSNTY

SEQ ID NO: 52 - protein, 72 aa, Artificial Sequence; identical to SEQ ID NO: 51
  Exendin analog

SEQ ID NO: 53 - protein, 399 aa, Artificial Sequence
  Zot from V. cholerae BX 330286
MSIFIHHGAPGSYKTSGALWLRLLPAIKSGRHIITNVRGLNLERMAKYLKMDVSDISIEFIDTDHPDGRLTMARFWHWARKDAFLFIDECGRIWPPRLTATNLKALDTPPDLVAEDRPESFEVAFDMHRHHGWDICLTTPNIAKVHNMIREAAEIGYRHFNRATVGLGAKFTLTTHDAANSGQMDSHALTRQVKKIPSPIFKMYASTTTGKARDTMAGTALWKDRKILFLFGMVFLMFSYSFYGLHDNPIFTGGNDATIESEQSEPQSKATAGNAVGSKAVAPASFGFCIGRLCVQDGFVTVGDERYRLVDNFDIPYRGLWATGHHIYKDTLTVFFETESGSVPTELFASSYRYKVLPLPDFNHFVVFDTFTAQALWVEVKRGLPIKTENDKKGLNSIF

SEQ ID NO: 54 - protein, 6 aa, Artificial Sequence
  Zot from V. cholerae BX 330286 (residues 288-293)
FCIGRL

SEQ ID NO: 55 - protein, 7 aa, Artificial Sequence
  Zot peptide or analog thereof
FXIGRLK

SEQ ID NO: 56 - protein, 7 aa, Artificial Sequence
  Zot peptide or analog thereof
FXIGRLK

SEQ ID NO: 57 - protein, 5 aa, Artificial Sequence
  linker
GPNGG

SEQ ID NO: 58 - protein, 5 aa, Artificial Sequence
  linker
KKKKK

SEQ ID NO: 59 - protein, 6 aa, Artificial Sequence
  linker
PPPPPP

(19) United States
(12) Patent Application Publication
(10) Pub. No.: US 2015/0111246 A1
(43) Pub. Date: Apr. 23, 2015

(54) SITE-SPECIFIC ENZYMATIC MODIFICATION OF EXENDINS AND ANALOGS THEREOF

(71) Applicants: Amylin Pharmaceuticals, LLC, San Diego, CA (US); AstraZeneca Pharmaceuticals LP, Wilmington, DE (US)

(72) Inventors: Manoj P. Samant, Burlington, MA (US); Lawrence D'Souza, San Diego, CA (US); Odile E. Levy, San Diego, CA (US); Soumitra S. Ghosh, San Diego, CA (US); Christopher J. Soares, La Jolla, CA (US)

(73) Assignees: ASTRAZENECA PHARMACEUTICALS LP; AMYLIN PHARMACEUTICALS, LLC

(21) Appl. No.: 14/396,225

(22) PCT Filed: Apr. 23, 2013
(86) PCT No.: PCT/US2013/037770

§ 371 Date: Oct. 22, 2014

Related U.S. Application Data
(60) Provisional application No. 61/637,393, filed on Apr. 24, 2012.

(51) Int. Cl. C07K 14/605; C12P 21/00

(52) U.S. Cl. CPC C07K 14/605; C12P 21/00
USPC 435/68.1

(57) ABSTRACT

There is provided inter alia a method of covalently attaching an amine derivatizing agent to a glutamine of a first polypeptide. The method includes adding an amine derivatizing agent, a transglutaminase a first polypeptide which includes a glutamine, and a co-solvent to a reaction mixture. The method further includes allowing the amine derivatizing agent to react with the glutamine of the first polypeptide in the reaction mixture to form an amide bond, thereby covalently attaching the amine derivatizing agent to the glutamine of the first polypeptide.

### CROSS-REFERENCES TO RELATED APPLICATIONS

The present application claims the benefit of U.S. Provisional Application No. 61/637,393 filed on Apr. 24, 2012, which is herein incorporated by reference in its entirety.

### BACKGROUND OF THE INVENTION

The present application relates to novel methods for forming covalent modifications of polypeptides, e.g., condensation of two or more polypeptides, covalent binding of peptidic or non-peptidic components to a polypeptide, and the like.

Covalent modification of peptides is a useful methodology for increasing the pharmaceutical benefit of peptides. For example, covalent modifications can increase peptide concentration in vivo, reduce immunogenicity, increase proteolytic stability, and the like. Accordingly, there is provided herein an enzymological method for covalent modification of a polypeptide using transglutamination via a transglutaminase (E.C. 2.3.2.13) for site-specific modification of a glutamine residue included in the polypeptide.

The use of transglutaminases in the covalent modification of polypeptides has been reported for a variety of polypeptides. See e.g., U.S. Published Patent Appl. No 2007/0105770. Without wishing to be bound by any theory, it is believed that the reaction catalyzed by transglutaminase can result in incomplete covalent attachment of polypeptide modified moieties and the production of a variety of contaminating species. Accordingly, there is a need for novel methods of polypeptide modification employing transglutamination which result in few contaminating species. Provided herein are methods and compositions addressing these and other needs in the art.

### BRIEF SUMMARY OF THE INVENTION

In a first aspect, there is provided a method of covalently attaching an amine derivatizing agent to a glutamine of a first polypeptide. The method includes adding an amine derivatizing agent, a transglutaminase, a first polypeptide which includes a glutamine, and a co-solvent to a reaction mixture. The method further includes allowing the amine derivatizing agent to react with the glutamine of the first polypeptide in the reaction mixture to form an amide bond, thereby covalently attaching the amine derivatizing agent to the glutamine of the first polypeptide.

### BRIEF DESCRIPTION OF THE DRAWINGS

FIG. 1. FIGS. 1A-1C depict RP-HPLC chromatograms of Exendin-4 in 20 mM Tris buffer, 5 mM CaCl₂, with no co-solvent (FIG. 1A), 5% propylene glycol (FIG. 1B), and 5% PEG 300 (FIG. 1C). See Example 1. FIGS. 1D-1F depict RP-HPLC chromatograms of reaction mixtures of FIGS. 1A-1C, respectively, after 18-hrs reaction with transglutaminase.

FIG. 2. FIG. 2 depicts time course of blood glucose in test subject tor 4-hrs after IP injection of vehicle or test compound (2 nmol/kg). See Example 3. Legend: Vehicle (gray box); Exendin-4 (closed triangle tip up); Cmpd 2-1 (closed diamond); Cmpd 2-2 (closed circle); Cmpd 2-3 (closed triangle tip down); Cmpd 2-4 (black box); Cmpd 2-5 (open triangle); Cmpd 2-8 (open diamond); Cmpd 2-9 (open circle).

FIG. 3. FIG. 3 depicts an RP-HPLC chromatogram of the reaction of Exendin-4 with N_{α}-octan-1-amido-lysine after 8-hrs under the transglutamination conditions described in Example 4.

FIG. 4. FIG. 4A depicts an RP-HPLC chromatogram at zero time for the reaction of Exendin-4 with a cholic acid derivative. See Example 5. FIGS. 4B depicts an RP-HPLC chromatogram at 8-hrs under the transglutamination conditions described in Example 5.

FIG. 5. FIG. 5 depicts an RP-HPLC chromatogram after 3-hrs reaction of pramlinitide with m-dPEG24-amine as described in Example 12.

FIG. 6. FIG. 6 depicts an RP-HPLC chromatogram showing solution phase chemical conjugation reaction on [Arg^{27]Exendin-}4 using EDC as activator show no product formation.

FIG. 7. FIG. 7 depicts an RP-HPLC chromatogram showing solution-phase chemical conjugation reaction on [Arg27]Exendin-4 using 4-PNP chloroformate as activator show some conjugated product.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is alkyl attached to the remainder of the molecule via an oxygen linker (—O—). An alkylthiol is an alkyl attached to the remainder of the molecule via a sulfur linker (—S—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂—, and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxaliny, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl respectively.

For brevity, the term “aryl” when used in combination with other terms (e.g., aryloxy, arylthioxy, arylalkyl and the like) includes both aryl and heteroaryl rings as defined herein. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl and the like).

The term “oxo, ” as used herein, means an oxygen that is double bonded to a carbon atom.

The term “alkylsulfonyl,” as used herein, means a moiety having the formula —S(O₂)—R′, where R′ is an alkyl group as defined above, R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided herein.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —CN, and —NO₂in a number ranging from zero to (2m′+1), where m′ is the total number of carbon atoms in such radical. R′, R″, R″′, and R″″ each preferably independently refer to hydrogen, substituted of unsubstituted heteroalkyl, substituted or unsubstituted cycloalkyl, substituted or unsubstituted heterocyloalkyl, substituted or unsubstituted aryl (e.g., aryl substituted with 1-3 halogens), substituted or unsubstituted alkyl, alkoxy, or thioalkoxy groups, or arylalkyl groups. When a compound described herein includes more than one R group, for example, each of the R groups is independently selected as are each R′, R″, R″′ and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR″′, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′, and R″″ are preferably independently selected from hydrogen, substituted or unsubstituted alkyl substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula —T—C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

Unless otherwise stated, a “substituent group” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
(i) oxo, —OH, —NH₂, —SH, —CN, —CF₃, —NO₂, halogen, unsubstituted alkyl, unsubstituted heteroalkyl, unsubstituted cycloalkyl, unsubstituted heterocyctoalkyl, unsubstituted aryl, unsubstituted heteroaryl, and
(ii) alkyl, heteroalkyl, cycloalkyl, heterocycloalkyl, aryl, and heteroaryl, substituted with at least one substituent selected from:
   (a) oxo, —OH, —NH₂, —SH, —CN, —CF₃, —NO₂halogen, unsubstituted alkyl, unsubstituted heteroalkyl, unsubstituted cycloalkyl, unsubstituted heterocycloalkyl, unsubstituted aryl, unsubstituted heteroaryl, and
   (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope contemplated herein.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope contemplated herein.

The compounds disclosed herein may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope contemplated herein.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As is customary in the art, square brackets (i.e., “[ ]”) in a peptidic compound name indicates substitution of the residue or chemical feature within the square brackets. For example, [¹⁴Leu]Exendin-4, [Leu¹⁴]Exendin-4, [¹⁴Leu]Ex-4, [Leu¹⁴]Ex-4, and [¹⁴L]Exendin-4 all refer to exendin-4 having leucine at position 14. The numeric position of an amino acid can be indicated by prepended or postpended numbers in a variety of ways routinely employed in the art. For example, the terms “¹⁴Leu,” “Leu14,” “14Leu,” “Leu¹⁴” and the like, are synonymous in referring to leucine at position 14. It is understood that “Ex-4” refers to Exendin-4. As customary in the art, amino acid residues can be referenced by one-letter or three-letter codes, e.g., “A,” “C” and “D” refer to Ala, Cys and Asp, respectively.

“Obesity” and “overweight” refer to mammals having a weight greater than normally expected, and may be determined by, e.g., physical appearance, body mass index (BMI) as known in the art, waist-to-hip circumference ratios, skinfold thickness, waist circumference, and the like. The Centers for Disease Control and Prevention (CDC) define overweight as an adult human having a BMI of 25 to 29.9; obese as an adult human having a BMI of 30.0 or higher, and extreme obesity as an adult human having a BMI greater than or equal to 40.0. Additional metrics for the determination of obesity exist. For example, the CDC states that a person with a waist-to-hip ratio greater than 1.0 is overweight.

“Lean body mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA) as known in the art.

“Mammal” refers to warm-blooded animals that generally have fur or hair, that give live birth to their progeny, and that feed their progeny with milk. Mammals include humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a human having type 2 diabetes. In one embodiment, the mammal is an obese diabetic mammal, e.g., an obese mammal having type 2 diabetes. The term “subject” in the contest of methods described herein refers to a mammal. Preferably the subject is a primate, and more preferably human.

“Fragment” in the context of polypeptides refers herein in the customary chemical sense to a portion of a polypeptide. For example, a fragment can result from N-terminal deletion or C-terminal deletion of one or more residues of a parent polypeptide, and/or a fragment can result from internal deletion of one or more residues of a parent polypeptide. “Fragment” in the context of an antibody refers to a portion of an antibody which can be linked to a biologically active molecule to modulate solubility, distribution within a subject, and the like. For example, exendin-4(1-30) describes a biologically active fragment of exendin-4 where the exendin C-terminal “tail” of amino acids 31-39 is deleted. The term “parent” in the context of polypeptides refers, in the customary sense, to a polypeptide which serves as a reference structure prior to modification, e.g., insertion, deletion and/or substitution. The term “conjugate” in the context of compounds described herein refers to covalent linkage between component polypeptides forming the compound. The term “fusion” in the contest of compounds described herein refers to covalent linkage between component polypeptides via either or both terminal amino or carboxy functional group of the peptide backbone. Compounds described herein can be synthetically or recombinants made. Typically, fusions are made using recombinant biotechnology, however, can also be made by chemical synthesis and conjugation methods.

“Analog” as used herein in the context of polypeptides refers to a compound that has insertions, deletions and/or substitutions of amino acids, or surrogates thereof including des-amino compounds, relative to a parent compound. An analog may have superior stability, solubility, efficacy, half-life, and the like. In some embodiments, an analog is a compound having at least 50%, for example 50%, 55%, 60%, 65%, 70%, 75%, 80%, 85%, 90%, 95%, 98%, or even higher, sequence identity to the parent compound. In a preferred embodiment the analog has from 1 to 5 amino acid modifications selected independently from any one or combination of an insertion, deletion, addition and substitution. In any of the embodiments herein, the exendin analog can have from 1 to 5 amino acid modifications selected independently from any one or combination of an insertion, deletion, addition and substitution, and preferably retains at least 50%, for example 50%, 55%, 60%, 65%, 70%, 75%, 80%, 85%, 90%, 95%, 98%, or even higher, sequence identity to the parent compound, and even more preferably at least 80%, 85%, 90%, 95%, 98%, or even higher, sequence identity to the parent compound, and preferably the parent compound is exendin-4, exendin-4(1-38), exendin-4(1-37), exendin-4(1-36), exendin-4(1-35), exendin-4(1-34), exendin-4(1-33), exendin-4(1-32), exendin-4(1-31), exendin-4(1-30), exendin-4(1-29) or exendin-4(1-28), and most preferably the parent compound is exendin-4. In one embodiment at least amino acids corresponding to positions 1, 4, 6, 7 and 9 of exendin-4 are those as in native exendin-4, and further the one to five modifications are conservative amino acid substitutions at positions other than positions 1, 4, 6, 7 and 9 of exendin-4. For example, in yet a further embodiment of the embodiments herein, an exendin analog retains the amino acid at least as found in position 3, 4, 6, 5, 7, 8, 9, 10, 11, 13, 15, 18, 19, 22, 23, 25, 26, and/or 30 of exendin-4, and further preferably has no more than 1 to 5 of the remaining positions substituted with another amino acid, most preferably a chemically conservative amino acid. In all of the analogs herein, any substitution or modification at positions 1 and/or 2 will retain resistance to DPP-IV cleavage while retaining or improving insulinotropic activity as is known in the art for exendin-4 analogs, such as desamino-histidyl-exendin-4. As customary in the art, the term “conservative” in the context of amino acid substitutions refers to substitution which maintains properties of charge type (e.g., anionic, cationic, neutral, polar and the like), hydrophobicity or hydrophilicity, bulk (e.g., van der Waals contacts and the like), and/or functionality (e.g., hydroxy, amine, sulfhydryl and the like). The term “non-conservative” refers to an amino acid substitution which is not conservative.

Amino acids, and surrogates thereof, for use as substituents in the analogs described herein are abbreviated as known in the art. Exemplary abbreviations include the following: Hpa (4-hydroxyphenylacetic acid); MetO (methioninine sulphoxide); Ahx (2-aminohexanoic acid); Sar (sarcosine); Nal (naphthylalanine). As customarily in the art, the prefix “D” in the context of an amino acid refers to the “D” stereoisomer thereof; the prefix “Me” refers to methylation, e.g., MeAhx, MeAsp, NMeAsp, MeDAsp, MePhe, NMePhe, and the like; the prefix “Et” refers to ethylation, e.g., EtPhe and the like.

“Derivative” in the context of a peptide disclosed herein refers to compounds which include a non-peptidic moiety covalent bound thereto, e.g., a linker between multiple peptides, polyethylene glycol, fatty acyl conjugate, and the like. Derivatives can be formed by bonding to any available site on the peptide, e.g., backbone or side chain bonding.

“Identity,” “sequence identity” and the like in the context of comparing two or more nucleic acids or polypeptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl Math. 2:482, by the homology alignment algorithm of Needleman & Wunsch, 1970, J. Mol. Biol. 48:443, by the search tor similarity method of Pearson & Lipman, 1988, Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See e.g. Current Protocols in Molecular Biology (Ausubel et al., eds. 1995 supplement)). Preferred examples of algorithms that are suitable for determining percent sequence identity and sequence similarity include the BLAST and BLAST 2.0 algorithms, which are described in Atschul et al., 1977, Nuci. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins of the indention. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence T is referred to as the neighborhood word score threshold (Altschul et al., Id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, M=−4 and a comparison of both strands. For amino acid sequences, the BLASTP program uses as defaults a wordlength of 3, and expectation (E) of 10, and the BLOSUM62 scoring matrix (see Henikoff & Henikoff. 1989, Proc. Natl. Acad. Sci. USA 89:10915) alignments (B) of 50, expectation (E) of 10, M=5, M=−4, and a comparison of both strands.

The terms “a,” “an,” or “a(n)”, when used in reference to a group of substituents herein, mean at least one. For example, where a compound is substituted with “an” alkyl or aryl, the compound is optionally substituted with at least one alkyl or at least one aryl. Where a moiety is substituted with an R substitute, the group may be referred to as “R-substituted.” Where a moiety is R-substituted, the moiety is substituted with at least one R substituent and each R substituent is optionally different. The term “about” in the context of a numeric value refers to ±10% of the numeric value.

The terms “peptide” and “polypeptide” as used described herein are synonymous.

As used herein, and as well-understood in the art, “treatment” is an approach for obtaining beneficial or desired results, including clinical results. “Treating,” “palliating,” or “ameliorating” a disease, disorder, or condition means that the extent, undesirable clinical manifestations of a condition, or both, of a disorder or a disease state are lessened and/or the time course of the progression is slowed (i.e., lengthened in time), as compared to not treating the disorder. For purposes of the methods disclosed herein, beneficial or desired clinical results include, but are not limited to, alleviation or amelioration of one or more symptoms, diminishment of extent of disorder, stabilized (i.e., not worsening) state of disorder, delay or slowing of disorder progression, amelioration or palliation of the disorder, and remission (whether partial or total), whether detectable or undetectable. “Treatment” can also mean prolonging survival as compared to expected survival if not receiving treatment. Further, treating does not necessarily occur by administration of one dose, but often occurs upon administration of a series of doses. Thus, a “therapeutically effective amount,” an amount sufficient to palliate, or an amount sufficient to treat a disease, disorder, or condition may be administered in one or more administrations. The therapeutically effective amount is one that provides the desired, described therapeutic effect when administered according to an embodiment described herein.

### II. Compounds

Polypeptides contemplated for use in the methods disclosed herein include amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or an analog, derivative or fragment thereof.

Amylins. Amylins, and analogs and fragments thereof suitable for use in the compounds and methods described herein include the compounds described in WO 2007/022123 (PCT/US2006/031724, filed Aug. 11, 2006), incorporated herein by reference and for all purposes. Amylin is a peptide hormone synthesized by pancreatic β-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin, as known in the art. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic analog of human amylin, reduces postprandial glucose excursions by suppressing inappropriately elevated postprandial glucagon secretion and slowing gastric emptying. The sequences of rat amylin, human amylin and pramlintide follow:

(SEQ ID NO: 1)
KCNTATCATQRLANFLVRSSNNLOGPVLPPTNVGSNTY;
(SEQ ID NO: 2)
KCNTATCATQRLANFLVHSSNNFGAILSSTNVGSNTY;
(SEQ ID NO: 3)
KCNTATCATQRLANFLNHSSNNFGPILPPTNVGSNTY.

Adrenomedullins (ADMs). Adrenomedullin (ADM) and analogs and fragments thereof suitable for use in the compounds and methods described herein include the compounds described in U.S. Pat. Nos. 8,076,288, 8,007,794, 7,928,060, and 7,879,794, each of which is incorporated herein by reference and for all purposes. ADM is a member of the calcitonin peptide family, first isolated in 1993 from human pheochromocytoma. See e.g., Kitamura, K., et al., 1993, Biochem. Biophys. Res. Commun. 192:553-560. ADM is generated from a 185 amino acid preprohormone with sequence MKLVSVALMYLGSLAFLGADTARLDVASEFRKKWN KWALSRGKRELRMSSSYPTGLADVKAGPAQTLIRPQDMKGASRSPEDSSPDAARIRV KRYRQSMNNFQGLRSFGCRFGTCTVQKLAHQIYQFTDKDKDNVAPRSKISPQGYGR RRRRSLPEAGPGRTLVSSKPQAHGAPAPPSGSAPHFL (SEQ ID NO:4) through consecutive enzymatic cleavage and amidation. This process culminates in the liberation of a 52 amino acid bioactive peptide with sequence YRQSMNNFQGLRSFGCRFGTCTVQKLAHQIYQFTDKD KDNVAPRSKISPQGY(SEQ ID NO:5).

Calcitonins (CTs). Calcitonin (CT) and analogs and fragments thereof suitable for use in the compounds and methods described herein include the compounds described in U.S. Pat. Nos. 8,076,291, 8,076,288, 7,928,060, and 7,879,794, each of which is incorporated herein by reference and for all purposes. Calcitonin is produced in and secreted from neuroendocrine cells in the thyroid, i.e., “C cells.” A well-studied action of salmon CT(1-32) (sequence: CSNLSTCVLGKLSQELHKLQTYPRTNTGSGTP, SEQ ID NO:6) is its effect on the osteoclast. In vitro effects of CT include the rapid loss of ruffled borders and decreased release of lysosomal enzymes. Ultimately, the inhibition of osteoclast functions by CT results in a decrease in bone resorption. However, neither a chronic reduction of serum CT in the case of thyroidectomy nor the increased serum CT found in medullary thyroid cancer appears to be associated with changes in serum calcium or bone mass. See e.g., Becker, et al., 2004, JCEM 89(4): 1512-1525; Sexton, et al., 1999, Current Medicinal Chemistry 6:1067-109; Kurihara et al., 2003, Hypertens Res. 26 Suppl:S 105-108.

Calcitonin Gene Related Protein (CGRP). Calcitonin Gene Related Protein (CGRP) and analogs, derivatives and fragments thereof suitable for use in the compounds and methods described herein include the compounds described in U.S. Pat. Nos. 5,124,314, 5,266,561, 5,677,279, each of which is incorporated herein by reference and for all purposes.

Intermedins. Intermedin (also known as Amylin Family Peptide 6, “AFP-6”) and analogs, derivatives and fragments thereof suitable for use in the compounds and methods described herein include the compounds described in U.S. Pat. Nos. 8,076,288, 7,928,060, 7,879,794, 7,671,023, each of which is incorporated herein by reference and for all purposes. An exemplary intermedin has the sequence TQAQLLRVGCVLGTCQVQNLSHRLWQLMGPA GRQDSAPVDPSSPHSY (SEQ ID NO:7). In vivo administration of intermedin leads to blood pressure reduction in both normal and spontaneously hypertensive rats, and in mice leads to suppression of gastric emptying and food intake. See e.g., Roh et al., 2004, J. Biol. Chem. 279:7264-7274.

Cholecystokinins (CCK). Cholecystokinins, and analogs and fragments thereof suitable for use in the compounds and methods described herein include the compounds described in U.S. Pat. No. 8,076,288 (filed Aug. 17, 2005), incorporated herein by reference and for all purposes. The cholecystokinins (CCKs) are peptide hormones of the central nervous and gastrointestinal systems responsible a variety of actions, including satiety and stimulation of digestion.

CCK is subject to post-translational modifications, including cleavage, of preprocholecystokinin (PPCCK), with sequence MNSGVCLCVLMAVLAAGALTQPVPPADPAGSGLQRAEEAPRRQLRVSQRTDGESR AHLGALLARYIQQARKAPSGRMSIVKNLQNLDPSHRISDRDYMGWMDFGRRSAEEY EYPS (SEQ ID NO:8).

Accordingly, post-translational products of preprocholecystokinin include CCK-58 (PPCCK₄₆₋₁₀₃), CCK-39 (PPCCK₆₅₋₁₀₃), CCK-33 (PPCCK₇₁₋₁₀₃), CCK-22 (PPCCK₈₂₋₁₀₃), CCK-12 (PPCCK₉₂₋₁₀₃), and CCK-8 (PPCCK₉₆₋₁₀₃). CCK-8 is the most abundant form of CCK in the human brain, while in the human intestine and circulation CCK-58, CCK-33, CCK-22 and CCK-8 are present in significant concentrations. These are C-terminally amidated. See e.g., Eberlein et al., 1988, Peptides 9:993-998; Miyasaka et al., 2001, J Clin Endocrinol Metab 86:251-258.

Leptins. The term “leptin” refers to a polypeptide hormone of the leptin family as known in the art, and analogs, fragments and derivatives thereof. Exemplary leptins suitable for use in the compounds and methods described herein include, but are not limited to, the compounds described in U.S. Pat. No. 5,594,101, U.S. Pat. No. 5,851,995, U.S. Pat. No. 5,691,309, U.S. Pat. No. 5,580,954, U.S. Pat. No. 5,554,727, U.S. Pat. No. 5,552,523, U.S. Pat. No. 5,559,208, U.S. Pat. No. 5,756,461, U.S. Pat. No. 6,309,853, published U.S. Patent application No. US: 2007/0020284, and PCT Published Application Nos. WO 96/23517, WO 96/005309, WO 98/28427, WO 2004/039832, WO 98/55139, WO 98/12224, and WO 97/02004, each of which is incorporated herein in its entirety and for all purposes. Methods to assay for leptin activities and biological responses in vitro and in vivo, including satiety, food intake inhibition activity and weight loss activity, are known in the art.

In human, the mature form of circulating leptin is a 146-amino acid protein that is normally excluded from the CNS by the blood-brain barrier (BBB) and the blood-CSF barrier. See, e.g., Weigle et al., 1995. J Clin Invest 96: 2065-2070. The sequence of human leptin including a 21-residue N-terminal signal sequence follows: MHWGTLCGFLWLWPYLFYVQ AVPIQKVQDDTKTLIKTIVTRINDISHTQSVSSKQKVTGLDFIPGLHPILTLSKMDQTL AVYQQILTSMPSRNVIQISNDLENLRDLLHVLAFSKSCHLPWASGLETLDSLGGVLEA SGYSTEVVALSRLQGSLQDMLWQLDLSPGC (SEQ ID NO:9). The sequence of mature human leptin form 1 having an N-terminal methionine residue (also known as Metreleptin) follows: MVPIQKVQDDTKTLIKTIVTRINDISHTQSVSSKQKVTGLDFIPGLHPILTLSKMDQTL AVYQQILTSMPSRNVIQISNDLENLRDLLHVLAFSKSCHLPWASGLETLDSLGGVLEA SGYSTEVVALSRLQGSLQDMLWQLDLSPGC (SEQ ID NO:10).

Leptin is the afferent signal in a negative feedback loop regulating food intake and body weight. The leptin receptor is a member of the cytokine receptor family. Leptin's anorexigenic effect is dependent on binding to homodimer of the Ob-Rb isoform of this receptor which encodes a long intra-cytoplasmic domain that includes several motifs for protein-protein interaction. Ob-Rb is highly expressed in the hypothalamus suggesting that this brain region is an important site of leptin action. Mutation of the mouse ob gene has been demonstrated to result in a syndrome that exbibits-pathophysiology that includes: obesity, increased body fat deposition, hyperglycemia, hyperinsulinemia, hypothermia, and impaired thyroid and reproductive functions in both male and female homozygous ob/ob obese mice (see e.g., Ingalis, et al., 1950. J Hered 41: 317-318. Therapeutic uses for leptin or leptin receptor include (i) diabetes (see, e.g., PCT Patent Applications WO 98/55139, WO 98/12224, and WO 97/02004); (ii) hematopoiesis (see, e.g., PCT Patent Applications WO 97/27286 and WO 98/18486); (iii) infertility (see, e.g., PCT Patent Applications WO 97/15322 and WO 98/36763); and (iv) tumor suppression (see, e.g., PCT Patent Applications WO 98/48831), each of which are incorporated herein by reference in their entirety.

Pancreatic Polypeptide Family. Another family of peptide hormones implicated in metabolic diseases and disorders and suitable for derivatization by the method disclosed herein is the pancreatic polypeptide family (“PPF”). Pancreatic polypeptide (“PP”) was discovered as a contaminant of insulin extracts and was named by its organ of origin rather than functional importance. See Kimmel et al., Endocrinology 83; 1323-30 (1968). PP is a 36-amino acid peptide containing distinctive structural motifs. A related peptide was subsequently discovered in extracts of intestine and named Peptide YY (“PYY”) because of the N- and C-terminal tyrosines. See Tatemoto, Proc. Natl. Acad. Sci. USA 79: 2514-8 (1982). A third related peptide was later found in extracts of brain and named Neuropeptide Y (“NPY”). See Tatemoto, 1982, Proc. Natl. Acad. Sci. USA 79: 5485-9; Tatemoto et al., 1982, Nature 296: 659-60. The sequences of human PP, PYY and NPY, respectively, follow:

(SEQ ID NO: 11)
APLEPVYPGDNATPEQMAQYAALKRRYINMLTRPRY-NH₂
(SEQ ID NO: 12)
YPIKPEAPGEDASPEELNRYYASLRHYLNLVTRQRY-NH₂
(SEQ ID NO: 13)
YPSKPDNPGEDAPAEDMARYYSALRHYINLITRQRY-NH₂.

Polypeptides of the pancreatic polypeptide family suitable for use in the compounds and methods described herein include the compounds described in U.S. Pat. Nos. 8,076,288 and 7,723,471, U.S. Patent Appl. Publ. Nos. 2010/0286365 and 2010/0099619, 2006/0293232, and PCT Published Appl. Nos. WO2005/077094 and WO2006/066024, each of which is incorporated herein by reference in its entirety and for all purposes.

Glucagon-like Peptide-1 (GLP-1). Glucagon-like Peptide-1 (GLP-1) and analogs, derivatives and fragments thereof suitable for use in the compounds and methods described herein include the compounds described in U.S. Pat. Nos. 6,506,724, 6,579,851, each of which is incorporated herein by reference and for all purposes.

GLP-1 Analogs and Derivatives. “GLP-1 receptor agonist” refers to compounds having GLP-1 receptor binding and activating activity. While such exemplary compounds include exendins, exendin analogs and exendin derivatives as described herein, it also includes GLP-1 and GLP-1 analogs such as GLP-1(7-37): HAEGTFTSDVSSYLEGQAAKEFIAWLVKGR-NH₂ (SEQ ID NO:14), GLP-1(7-37) analogs, and GLP-1(7-37) derivatives.

“GLP-1 analogs”, as differentiated from “exendin analogs,” refers to peptides derived from GLP-1(7-37), have structural similarity to GLP-1(7-37) of greater than 70% amino acid identity, and that elicit a biological activity similar to that of GLP-1(7-37), when evaluated by art-known measures such as receptor binding assays or in viva blood glucose assays as described, e.g., by Hargrove et al., Regulatory Peptides, 141:113-119 (2007), the disclosure of which is incorporated by reference herein and for all purposes. In one embodiment, the term “GLP-1(7-37) analog” refers to a peptide that has an amino acid sequence with 1, 2, 3, 4, 5, 6, 7 or 8 amino acid substitutions, insertions, deletions, or a combination of two to eight thereof, when compared to the amino acid sequence of GLP-1(7-37). In one embodiment, the GLP-1(7-37) analog is GLP-1(7-36)-NH₂. GLP-1(7-37) analogs include the amidated forms, the acid form, the pharmaceutically acceptable salt form, and any other physiologically active form of the molecule, such as chemically modified forms (e.g. via pegylation) or fusions (e.g. with Fc or albumin).

Exemplary GLP-1(7-37)-and GLP-1(7-37) analogs include GLP-1(7-37); GLP-1(7-36)-NH₂; liraglutide (VICTOZA® from Novo Nordisk); albiglutide (SYNCRIA® from GlaxoSmithKline); taspoglutide (Hoffman La-Roche); dulaglutide (also known LY2189265; Eli Lilly and Company); LY2428757 (Eli Lilly and Company); semaglutide (Novo Nordisk); desamino-His⁷, Arg²⁶, Lys³⁴(N^{ε}-(γ-Glut(N-α-hexadecanoyl)))-GLP-1(7-37); desamino-His⁷, Arg²⁶,Lys³⁴(N^{ε}-octanoyl)-GLP-1(7-37); Arg^{26,34}, Lys³⁸(N^{ε}-(ω-carboxypentadecanoyl))-GLP-1(7-38); Arg^{26,34},Lys³⁶(N^{ε}(γ-Glu(N-α-hexadecanoyl)))-GLP-1(7-36); Aib^{8,35},Arg^{26,34},Phe³¹-GLP-1(7-36)); compounds which include the formula HXaa_{g}EGTFTSDVSSYLEXaa₂₂Xaa₂₃AAKEFIXaa₃₀ WLXaa₃₃Xaa₃₄Xaa₃₇, wherein Xaa_{g} is A, V, or G, Xaa₂₂ is G, K, of E, Xaa₂₃ is Q or K, Xaa₃₀ is A or E, Xaa₃₃ is V or K, Xaa₃₄ is K, N, or R, Xaa₃₆ is R or G, and Xaa₃₇ is G, H, P, or absent (SEQ ID NO:15); Arg³⁴-GLP-1(7-37); Glu³⁰-GLP-1(7-37); Lys²²-GLP-1(7-37); Gly^{8,36},Glu²²-GLP-1(7-37); Val⁸,Glu²²,Gly³⁶-GLP-1(7-37); Gly^{8,36},Glu²²,Lys³³, Asn³⁴-GLP-1(7-37); Val⁸,Glu²²,Lys³³,Asn³⁴,Gly³⁶-GLP-1(7-37); Gly^{8,36},Glu²²,Pro³⁷-GLP-1(7-37); Val⁸,Glu²²,Gly³⁶Pro³⁷-GLP-1(7-37); Gly^{8,36},Glu²²,Lys³³,Asn³⁴,Pro³⁷-GLP-1(7-37); Val⁸,Glu²²,Lys³³,Asn³⁴,Gly³⁶,Pro³⁷-GLP-1(7-36); Val⁸,Glu²²,Gly³⁶-GLP-1(7-36); Val⁸,Glu²² ,Asn³⁴,Gly³⁶-GLP-1(1-36); Gly^{8,36},Glu²² ,Asn³⁴-GLP-1(7-36). Each of the GLP-1(7-37) and GLP-1(7-37) analogs may optionally be antidated.

GLP-1 receptor agonist compounds also includes oxyntomodulin, glucagon or GLP-1 analogs modified to bind and activate the GLP-1 receptor while either having glucagon receptor agonism or GIP receptor agonsim. Exemplary compounds include ZP2929 (Zealand Pharma) and those disclosed in patent application publication WO/2010/070253 and long acting acylated variants such as the peptides and acylated peptides disclosed in patent application publication WO/2011/006497; those GLP-1 receptor/glucagon receptor agonists disclosed in publications WO/2011/075393, WO/2009/155257 and WO/2009/155258, including their acylated and pegylated forms; those GLP-1 receptor/GIP receptor agonists, such as MAR701 (Marcadia), and those disclosed in WO2010011439A2, including their acylated and pegylated forms.

In one embodiment, the GLP-1(7-37) or GLP-1(7-37) analogs are covalently linked (directly or by a linking group) to an Fc portion of an immunoglobulin (e.g., IgG, IgE, IgG, and the like). For example, any one of the GLP-1 compounds disclosed above can be covalently linked to the Fc portion of an immunoglobulin, e.g. including the sequence of: AESKYGPPCPPCPAPXaa₁₆Xaa₁₇Xaa₁₈GGPSVFLFPPKPKDTLMISRTPEVTCVVVDVSQ EDPEVQFNWYVDGVEVHNAKTKPREEQFXaa₈₀STYRVVSVLTVLHQDWLNGKEYK CKVSNKGLPSSIEKTISKAKGQPREPQVYTLPPSQEEMTKNQVSLTCLVKGFYPSDIA VEWESNGQPENNYKTTPPVLDSDGSFFLYSRLTVDKSRWQEGNVFSCSVMHEALHN HYTQKSLSLSLGXaa₂₃₀, wherein Xaa₁₆ is P or E; Xaa₁₇ is F, V or A; Xaa₁₈ is L, E or A; Xaa₈₀ is N or A; and Xaa₂₃₀ is K or absent (SEQ ID NO: 16). The linking group may be any chemical moiety (e.g., amino acids and/or chemical groups), including a peptide bond (e.g. recombinant fusion). In one embodiment, the linking group is (-GGGGS-)ₓ where x is 1, 2, 3, 4, 5 or 6; preferably 2, 3 or 4; more preferably 3. In one embodiment, the GLP-1(7-37) analog covalently linked to the Fc portion of an immunoglobulin, includes the amino acid sequence:

(SEQ ID NO: 17)
HGEGTGTSDVSSYLEEQAAKEFIAWLVKGGGGGGGSGGGGSGGGGSAE
SKYGPPCPPCPAPEAAGPSVFLFPPKPKDTLMISRTPEVTCVVVDVSQ
EDPEVQFNWYVDGVEVHNAKTKPREEQFNSTYRVVSVLTVLHQDWLNG
KEYKCKVSNKGLPSSIEKTISKAKGQPREPQVYTLPSQEEMTKNQVSL
TCLVKGFYPSDIAVEWESNGQPENNYKTTPPVLDSDGSFFLYSRLTVD
KSRWQEGNVFSCSVMHEALHNHYTQKSLSLSLG.

In another embodiment, the GLP-1(7-37) or GLP-1(7-37) analog may be covalently linked (directly or through a linking group) to one or two polyethylene glycol molecules. For example, a GLP-1(7-37) analog may include the amino acid sequence: HXaa₈EGTFTSDVS SYLEXaa₂₂QAAKEFIAWLXaa₃₃KGGPSSGAPPPC₄₅C₄₆-Z, wherein Xaa₈ is: D-Ala, G, V, L, I, S or T; Xaa₂₂ is G, E, D or K; Xaa₃₃ is: V or I; and Z is OH or NH₂, and, optionally, wherein (i) one polyethylene glycol moiety in covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆. In one embodiment, the GLP-1(7-37) analog is HVEGTFTSDVSSYLEEQAAKEFIAWLIKGGPSSGAPPPC₄₅C₄₆-NH₂. and, optionally, wherein (i) one polyethylene glycol moiety is covalently attached to C₄₅. (ii) one polyethylene glycol moiety is covalently attached to C₄₆, or (iii) one polyethylene glycol moiety is attached to C₄₅ and one polyethylene glycol moiety is attached to C₄₆.

In one embodiment, including those GLP-1 analogs described above, the GLP-1 analog is a peptide derived from GLP-1(1-37) that has at least 80% sequence identity to GLP-1(7-37); at least 85% sequence identity to GLP-1(7-37); at least 90% sequence identity to GLP-1(7-37); or at least 95% sequence identity to GLP-1(7-37).

Glucagon-like Peptide-2 (GLP-2). Glucagon-like Peptide-2 (GLP-2) and analogs, derivatives and fragments thereof are suitable tor use in the compounds and methods described herein. GLP-2 is a 33 amino acid peptide having sequence: HADGSFSDEMNTILDNLAARD FINWLIQTKITD (SEQ ID NO:18). In mammals, GLP-2 is liberated from proglucagon in the intestine and brain but not in pancreas, as a result of cell-specific expression of prohormone convertases in gut endocrine cells (Dhanvantari et al., Mol. Endocrinol, 10: 342-355 (1996); Rothenberg et al., Mol. Endocrinol. 10: 334-341. (1996); Damholt et al., Endocrinology 140: 4800-4808 (1999); Hoist, Trends Endocrinol Metab, 10: 229-235 (1999)). Analysis of rat and human plasma using a combination of high-performance liquid chromatography and site-specific GLP-2 antisera reveals the presence of two principal circulating molecular forms, GLP-2₁₋₃₃ and GLP-2₂₋₃₃ (Hartmann et al., Supra; Brubaker et al., Endocrinol. 138: 4837-4843 (1997); Hartmann et al., L Clin. Endocrinol. Metab. 85: 2884-2888 (2000)). GLP-2₁₋₃₃ is cleaved in vivo by the protease dipeptidyl peptidase IV (DPP IV), which removes the first two residues, histidine and alanine (HA). The resulting peptide GLP-2₃₋₃₃ is essentially inactive.

Oxyntomodulin (OXM). Oxyntomodulin (OXM), and analogs, derivatives and fragments thereof are suitable for use in the compounds and methods described herein. OXM (also known as “glucagon-37”) is a 37-residue polypeptide including the 29-residue sequence of glucagon and an 8-residue C-terminal extension, with human sequence HSQGTFTSDYSKYLDSRRAQDFVQWLMNTKRNKNNIA (SEQ ID NO:19).

Natriuretic peptides. Natriuretic peptides and analogs, derivatives and fragments thereof suitable for use in the compounds and methods described herein include the compounds described in U.S. Pat. No. 8,076,288, incorporated herein by reference and for all purposes. The terms “natriuretic peptides” and the like refer to a family of hormones that include atrial natriuretic peptide (ANP), brain natriuretic peptide (BNP), and C-type natriuretic peptide (CNP). They are synthesized as three distinct precursor preprohormones, each encoded by separate genes having distinct sites of synthesis and mechanisms of regulation. Exemplary sequences of natriuretic peptides include human natriuretic peptides A preprotein (NCBI locus NP_{—}006163) (MSSFSTTTVSFLLLLAFQLLGQTRANPMYNAVSNADLMDFKNLLDHLEEKMPLEDE VVPPQVLSEPNEEAGAALSPLPEVPPWTGEVSPAQRDGGALGRGPWDSSDRSALLKS KLRALLTAPRSLRRSSCFGGRMDRIGAQSGLGCNSFRY) (SEQ ID NO:20); human ANP (SLRRSSCFGGRMDRIGAQSGLGCNSFRY) (SEQ ID NO:21); rat BNP (NCBI locus NP_{—}113733) (MDLQKVLPQMILLLFLNLSPLGGHSHPLGSPSQSPEQSTMQKLLELIREK SEEMAQRQLSKDQGPTKELLKRVLRSQDSAFRIQERLRNSKMAHSSSCFGQKIDRIG AVSRLGCDGLRLF) (SEQ ID NO:22); rat BNP (NSKMAHSSSCFGQKIDRIGAVSRLGCD GLRLF) (SEQ ID NO:23); porcine C-type natriuretic peptide precursor (NCBI locus NP_{—}001008482) (MHLSQLLACALLLTLLSLRPSEAKPGAPPKVPRTPPGEEVAEPQAA GGGQKKGDKTPGGGGANLKGDRSRLLRDLRVDTKSRAAWARLLHEHPNARKYKG GNKKGLSKGCFGLKLDRIGSMSGLGC) (SEQ ID NO:24); and porcine CNP (GLSKGCFGLKLDRIGSMSGLGC) (SEQ ID NO:25).

Urocortins. By “urocortin” is meant a human urocortin peptide hormone or species variants thereof in any physiological form. More particularly, there are three human urocortins; Ucn-1, Ucn-2 and Ucn-3. For example, human urocortin 1 has the formula: Asp-Asn-Pro-Ser-Leu-Ser-Ile-Asp-Leu-Thr-Phe-His-Leu-Leu-Arg-Thr-Leu-Leu-Glu-Leu-Ala-Arg-Thr-Gln-Ser-Gln-Arg-Glu-Arg-Ala-Glu-Gln-Asn-Arg-Ile-Ile-Phe-Asp-Ser-Val-NH2 (SEQ ID NO:26). Rat-derived urocortin is identical but for 2 substitutions: Asp² for Asn² and Pro⁴ for Ser4. Human Ucn-2 has the sequence Ile Val Leu Ser Leu Asp Val Pro Ile Gly Leu Leu Gln Ile Leu Leu Glu Gln Ala Arg Ala Arg Ala Ala Arg Glu Gln Ala Thr Thr Asn Ala Arg Ile Leu Ala Arg Val Gly His Cys (SEQ ID NO:27); Human Ucn-3 has the sequence Phe Thr Leu Ser Leu Asp Val Pro Thr Asn Ile Met Asn Leu Leu Phe Asn Ile Ala Lys Ala Lys Asn Leu Arg Ala Gln Ala Ala Ala Asn Ala His Leu Met Ala Gln Ile (SEQ ID NO:28). Ucn-3 is preferably in amide form. Further urocortins and analogs are described in the literature, for example in U.S. Pat. No. 6,214,797. Urocortins Ucn-2 and Ucn-3, which retain the food-intake suppression and antihypertensive/cardioprotective/inotropic properties, find particular use in the hybrids of the invention. Stresscopin (Ucn-3) and Stresscopin-related peptide (Ucn 2), named for their ability to suppress the chronic HPA activation following a stressful stimulus such as dieting/fasting, are specific for the CRF type 2 receptor and do not activate CRF-R1 which mediates ACTH release. Hybrids comprising a urocortin, e.g., Ucn-2 or Ucn-3, are particularly useful for vasodilation and thus for cardiovascular uses as described herein, e.g., CHF. Urocortin containing hybrids of the invention find particular use in treating or preventing conditions associated with stimulating ACTH release, hypertension due to vasodilatory effects, inflammation mediated via other than ACTH elevation, hyperthermia, appetite disorder, congestive heart failure, stress, anxiety, and psoriasis. Such compounds are also useful for an antiproliferative effect, such as for treating or preventing cancers or tumor growth. Of particular interest are urocortin peptide hormone module combined with a natriuretic peptide module, amylin family, an exendin family, or a GLP1 family module to provide an enhanced cardiovascular benefit, e.g. treating CHF, as by providing a beneficial vasodilation effect.

Neuromedin family peptides. Neuromedin and analogs and fragments thereof suitable for use in the compounds and methods described herein include the compounds described in U.S. Pat. Nos. 8,076,288 and 7,622,260, each of which is incorporated herein by reference and for all proposes. The term “neuromedin” refers to the neuromedin family of peptides including neuromedin U and S peptides, and the active hormone sequences thereof. For example, the native active human neuromedin U peptide hormone is neuromedin-U25: Phe Arg Val Asp Glu Glu Phe Gln Ser Pro Phe Ala Ser Gln Ser Arg Gly Tyr Phe Leu Phe Arg Pro Arg Asn (SEQ ID NO:29), particularly in the amide form. Pig U25 has the sequence: FKVDEEFQGPIVSQNRRYFLFRPRN (SEQ ID NO:30), particularly its amide form. Other neuromedin U family members include the following listed as their SWISS-PROT designations and entry numbers: NEUU_CANFA (P34962), NEUU_CAVPO (P34966), NEUU_CHICK (P34963) NEUU_HUMAN (P48645), NEUU_LITCE (PS1872), NEUU_MOUSE (Q9QXK8), NEUU_PIG (P34964), NEUU_RABIT (P34965), NEUU_RANTE (P20056), and NEUU_RAT (P12760). Of particular interest are their processed active peptide hormones and analogs, derivatives and fragments thereof. Included in the neuromedin U family are various truncated or splice variants, e.g., FLFHYSKTQKLGKSNVVEELQSPFASQSRGYFLFRPRN (SEQ ID NO:31). Exemplary of the neuromedin S family is human neuromedin S with the sequence ILQRGSGTAAVDFTKKDHTATWGRPFFLFRPRN (SEQ ID NO:32), particularly its amide form. Hybrids of the invention having neuromedin module will an anorexigenic effect, and thus have beneficial value in treating obesity, diabetes, reducing food intake, and other related conditions and disorders as described herein. Of particular interest are neuromedin modules combined with an amylin family peptide, an exendin peptide family or a GLP1 peptide family module.

Exendins. The exendins are peptides found in the salivary secretions of the Gila monster and the Mexican Bearded Lizard, which reptiles are endogenous to Arizona and Northern Mexico. Exendin-3 is present in the salivary secretions of Heloderma horridum (Mexican Beaded Lizard), and exendin-4 is present in the salivary secretions of Heloderma suspectum (Gila monster). See Eng et al, 1990, J. Biol. Chem., 265:20259-62; Eng et al, 1992, J. Biol. Chem., 267:7402-7405. The sequences of exendin-3 and exendin-4, respectively, follow:

(SEQ ID NO: 33)
HSDGTFTSDLSKQMEEEAVRLFIEWLKNGGPSSGAPPPS-NH₂;
(SEQ IS NO: 34)
HGEGTFTSDLSKQMEEEAVRLFIEWLKNGGPSSGAPPPS-NH₂.

Hargrove et al. (Regulatory Peptides, 2007, 141:113-119) reported an exendin-4 peptide analog that is a full-length C-terminally amidated exendin-4 peptide analog with a single nucleotide difference at position 14 compared to native exendin-4 (i.e., [¹⁴Leu]exendin-4) with sequence:

(SEQ ID NO: 35)
HGEGTFTSDLSKQLEEEAVRLFIEWLKNGGPSSGAPPPS-NH₂.

Another exendin-4 peptide analog is a chimera of the first 32 amino acids of exendin-4 having amino acid substitutions at positions 14 and 28 followed by a 5 amino acid sequence from the C-terminus of a non-mammalian (frog) GLP1, having sequence:

(SEQ ID NO: 36)
HGEGTFTSDLSKQLEEEAVRLFIEWLKQFFPSKEHS;

Also known in the art are C-terminally truncated, biologically active forms of exendin-4, such as exendin-4(1-28), exendin-4(1-29) and exendin-4(1-30) and their amidated forms. All of these exendin analogs are suitable as components of the combinations and co-administrations of compounds contemplated herein. Further particularly preferred analogs are desaminohistidyl exendin-4, dimethyl histidyl exendin-4 (where N-terminal amine is replaced with two —CH3 groups), beta-hydroxyl-imidazopropionyl exendin-4 (where the N-terminal amine is replaced with a hydroxyl), imidazopropionyl exendin-4, and imidazoacetyl-exendin-4 (where His1 alpha carbon and N-terminal amine are deleted), and their Leu14 analogs.

It is understood that in any embodiment a C-terminal amide, or other C-terminal capping moiety can be present in compounds described herein. Exendin-4 (or exenatide) by definition contains a C-terminal amide, however, its C-terminal non-amidated form is active and contemplated herein for use as or as part of exendin compound, as are the C-terminal non-amidated forms of the other exendins described herein.

Although the exendins have some sequence similarity to several members of the glucagon-like peptide (GLP) family, with the highest homology (53%) being to GLP-1(7-36)NH₂ (HAEGTFTSDVSSYLEGQAAKEFIAWLVKGR, SEQ ID NO:37, Goke et al, 1993, J. Biol Chem., 268:19650-55), also sometimes referred to as “GLP-1” and which has an insulinotropic effect stimulating insulin secretion from pancreatic beta-cells, exendins are not GLP-1 homologs.

Pharmacological studies have led to reports that exendin-4 can act at GLP-1 receptors in-vitro on certain insulin-secreting cells, however, it has also been reported that exendin-4 may act at receptors not acted upon by GLP-1. Further, exendin-4 shares some but not all biological properties in vivo with GLP-1, and it has a significantly longer duration of action than GLP-1. Based on their insulinotropic activities, the use of exendin-3 and exendin-4 for the treatment of diabetes mellitus and the prevention of hyperglycemia has been proposed (Eng, U.S. Pat. No. 5,424,286, incorporated herein by reference in its entirety and for all purposes), and in fact, exendin-4 has been approved in the United States and in Europe for use as a therapeutic for treating type 2 diabetes.

Indeed, without wishing to be bound by any theory, it is believed that exendins are not the species homolog of mammalian GLP-1 as was reported by Chen & Drucker who cloned the exendin gene from the Gila monster (J. Biol. Chem. 272:4108-15 (1997)). Without wishing to be bound by any theory, it is believed that the observation that the Gila monster also has separate genes for proglucagons (from which GLP-1 is processed) that are more similar to mammalian proglucagon than exendin suggests that exendins are not merely species homologs of GLP-1.

Methods for regulating gastrointestinal motility using exendin agonists are described in U.S. Pat. No. 6,858,576 (i.e., based on U.S. application Ser. No. 08/908,867 filed Aug. 8, 1997, which is a continuation-in-part of U.S. application Ser. No. 08/694,954 filed Aug. 8, 1996). Methods for reducing food intake using exendin agonists are described in U.S. Pat. No. 6,956,026 (i.e., based on U.S. application Ser. No. 09/003,869, filed Jan. 7, 1998, which claims the benefit of U.S. Application Nos. 60/034,905 filed Jan. 7, 1997, 60/055,404 filed. Aug. 7, 1997, 60/065,442 filed Nov. 14, 1997, and 60/066,029 filed Nov. 14, 1997.

Novel exendin agonist compounds useful in the compounds and methods described herein are described in WO 99/07404 (i.e., PCT/US98/16387 filed Aug. 6, 1998), in WO 99/25727 (i.e., PCT/US98/24210, tiled Nov. 13, 1998), in WO 99/25728 (i.e., PCT/US98/24273, filed Nov. 13, 1998), in WO 99/40788, in WO 00/41546, and in WO 00/41548, which are incorporated herein by reference and for all purposes along with their granted U.S. patent counterparts. Methods to assay for exendin activities in vitro and in vivo, as known in the art, including insulinotropic, food intake inhibition activity and weight loss activity, are described herein and also in the above references and other references recited herein.

Certain exemplary exendins, exendin agonists, and exendin analog agonists include: exendin fragments exendin-4(1-30) (His Gly Glu Gly Thr Phe Thr Ser Asp Leu Ser Lys Gln Met Glu Glu Glu Ala Val Arg Leu Phe Ile Glu Trp Leu Lys Asn Gly Gly) (SEQ ID-NO:38); exendin-4(1-28), exendin-4(1-29), exendin-4(1-30), exendin-4(1-3l) and exendin-4(1-32). Analogs include substitution at the ¹⁴Met position (i.e., ¹⁴Met) with a non-oxidizing amino acid such as leucine. Examples include [¹⁴Leu]exendin-4, [¹⁴Leu]exendin-4(1-30), [¹⁴Leu]exendin-4(1-28) and [¹⁴Leu, ²⁵Phe]exendin-4. In any and each of the exendin analogs and formulas described above, specifically contemplated are analogs thereof further including a replacement for the histidine corresponding to position 1 made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl, dimethyl histidyl, imidazopropionyl, and imidazoacetyl.

Exendin analog agonists for use in the methods and compositions described herein include compounds including the structure of Formula (1a) following;

(SEQ ID NO: 39)
Xaa₁ Xaa₂ Xaa₃ Gly Thr Xaa₆ Xaa₇ Xaa₈ Xaa₉ Xaa₁₀
Ser Lys Gln Xaa₁₄ Glu Glu Glu Ala Val Arg Leu
Xaa₂₂ Xaa₂₃ Xaa₂₄ Xaa₂₅ Leu Lys Asn Gly Gly Xaa₃₁
Ser Ser Gly Ala Xaa₃₆ Xaa₃₇ Xaa₃₈ Xaa₃₉

wherein Xaa₁ is His, Arg or Tyr; Xaa₂ is Ser, Gly, Ala or Thr; Xaa₃ is Asp or Glu; Xaa₆ is Phe, Tyr or naphthylalanine; Xaa₇ is Thr or Ser; Xaa₈ is Ser or Thr; Xaa₉ is Asp or Glu; Xaa₁₀ is Leu, Ile, Val, pentylglycine or Met; Xaa₁₄ is Leu, Ile, pentylglycine, Val or Met; Xaa₂₂ is Phe, Tyr or naphthylalanine; Xaa₂₃ is Ile, Val, Leu, pentylglycine, tert-butylglycine or Met; Xaa₂₄ is Glu or Asp; Xaa₂₅ is Trp, Phe, Tyr, or naphthylalanine; Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine or absent; and Xaa₃₉ is Ser, Thr or Tyr or absent. Optionally, the C-terminus of the peptide is amidated. In any and each of the exendin analogs and formulas described above, specifically contemplated are analogs thereof further including a replacement for the histidine corresponding to position 1 made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl, dimethyl histidyl, imidazopropionyl, and imidazoacetyl.

Exendin analog agonists for use in the methods and compositions described herein include those described in U.S. Pat. No. 7,223,725 (incorporated herein by reference and for all purposes), such as compounds of Formula (II) following:

(SEQ ID NO: 40)
Xaa₁ Xaa₂ Xaa₃ Gly Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala Xaa₁₉
Xaa₂₀ Xaa₂₁ Xaa₂₂ Xaa₂₃ Xaa₂₄ Xaa₂₅ Xaa₂₆ Xaa₂₇
Xaa₂₈.

In Formula (II), Xaa₁is His, Arg or Tyr; Xaa₂ is Ser, Gly, Ala or Thr; Xaa₃ is Ala, Asp or Glu: Xaa₅ is Ala or Thr; Xaa₆ is Ala, Phe, Tyr; Xaa₇ is Thr or Ser; Xaa₈ is Ala, Ser or Thr; Xaa₉ is Asp or Glu; Xaa₁₀ is Ala, Leu, Ile, Val or Met; Xaa₁₁ is Ala or Ser; Xaa₁₂ is Ala or Lys; Xaa₁₃ is Ala or Gln; Xaa₁₄ is Ala, Leu, Ile, Val or Met; Xaa₁₅ is Ala of Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Ala, Phe, Tyr; Xaa₂₃ is Ile, Val, Leu, or Met; Xaa₂₄ is Ala, Glu or Asp; Xaa₂₅ is Ala, Trp, Phe, Tyr; Xaa₂₆ is Ala or Leu; Xaa₂₇ is Ala or Lys; Xaa₂₈ is Ala or Asn. In any embodiment of Formula (II), the C-terminus of the peptide is optionally modified by -Z₁ which is —OH or —NH₂, or the C-terminus of the peptide further includes Gly-Z₂, Gly-Gly-Z₂, Gly-Gly-Xaa₃₁-Z₂, Gly-Gly-Xaa₃₁Ser-Z₂, Gly-Gly-Xaa₃₁-Ser-Ser-Z₂, Gly-Gly-Xaa₃₁-Ser-Ser-Gly-Z₂, Gly-Gly-Xaa₃₁-Ser-Ser-Gly-Ala-Z₂, Gly-Gly-Xaa₃₁Ser-Ser-Gly-Ala-Xaa₃₆-Z₂, Gly-Gly-Xaa₃₁-Ser-Ser-Gly-Ala-Xaa₃₆-Xaa₃₇-Z₂, or Gly-Gly-Xaa₃₁Ser-Ser-Gly-Ala-Xaa₃₆-Xaa₃₇-Xaa₃₈-Z₂; Xaa₃₁, Xaa₃₆, Xaa₃₇ and Xaa₃₈ are independently Pro or are absent; and the C-terminus of the peptide is modified by -Z₂, wherein -Z₂ is —OH or —NH₂. In any and each of the exendin analogs and formulas described above, specifically contemplated are analogs thereof further including a replacement for the histidine corresponding to position 1 made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl, dimethyl histidyl exendin-4, imidazopropionyl, or imidazoacetyl. Further specifically contemplated herein are exendin analogs described herein wherein a replacement for the glycine at Xaa₂ is made with any of D-Ala, Val, Leu, Lys, Aib (aminoisobutyric acid), (1-amino cyclopropyl)carboxylic acid, (1-aminocyclobutyl)carboxylic acid, 1-aminocyclopentyl) carboxylic acid, (1-aminocyclohexyl)carboxylic acid, (1-aminocycloheptyl)carboxylic acid, or (1-amino cycooctyl)carboxylic acid.

According to one embodiment, exemplary compounds include those of the above formula wherein: Xaa₁ is His or Arg; Xaa₂ is Gly or Ala; Xaa₃ is Asp or Glu; Xaa₅ is Ala or Thr; Xaa₆ is Ala or Phe; Xaa₇ is Thr or Ser; Xaa₈ is Ala, Ser or Thr; Xaa₉ is Asp or Glu; Xaa₁₆ is Ala, or Leu; Xaa₁₁ is Ala or Ser; Xaa₁₂ is Ala or Lys: Xaa₁₃ is Ala or Gln; Xaa₁₄ is Ala or Leu; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe; Xaa₂₃ is Ile, Val; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly-Gly-Z₂, Gly-Gly-Xaa₃₁-Z₂, Gly-Gly-Xaa₃₁-Ser-Z₂, Gly-Gly-Xaa₃₁-Ser-Ser-Z₂, Gly-Gly-Xaa₃₁-Ser-Ser-Gly-Z₂, Gly-Gly-Xaa₃₁-Ser-Ser-Gly-Ala-Z₂, Gly-Gly-Xaa₃₁-Ser-Ser-Gly-Ala-Xaa₃₆-Z₂, Gly-Gly-Xaa₃₁-Ser-Ser-Gly-Ala-Xaa₃₆-Xaa₃₇-Z₂, Gly-Gly-Xaa₃₁-Ser-Ser-Gly-Ala-Xaa₃₆-Xaa₃₇-Xaa₃₈-Z₂; Xaa₃₁, Xaa₃₆, Xaa₃₇ and Xaa₃₈ being independently Pro or is absent and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. In any and each of the exendin analogs described above, specifically contemplated are those wherein a replacement for the histidine corresponding to position 1 is made with any of D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine. Further specifically contemplated herein are exendin analogs described herein wherein a replacement for the glycine at Xaa₂ is made with any of D-Ala, Val, Leu, Lys, Aib, (1-aminocyclopropyl)carboxylic acid, (1-amino cyclobutyl)carboxylic acid, 1-aminocyclopentyl)carboxylic acid, (1-aminocyclohexyl)carboxylic acid, (1-amino cycloheptyl)carboxylic acid, or (1-aminocyclooctyl)carboxylic acid. In any and each of the exendin analogs and formulas described above, specifically contemplated are analogs thereof further including a replacement for the histidine corresponding to position 1 made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl dimethyl histidyl, imidazopropionyl, or imidazoacetyl.

Other exemplary compounds include those set forth in WO 99/25727 identified therein as compounds 2-23. According to another embodiment, provided are compounds where Xaa₃₄ is Leu, Ile, or Val more preferably Leu, and/or Xaa₂₅ is Trp, Phe or Tyr, more preferably Trp or Phe. These compounds will be less susceptive to oxidative degradation, both in vitro and in vivo, as well as during synthesis of the compound.

Additional examples of exendin analogs suitable for use in the present fusion polypeptides include those described in U.S. Pat. No. 6,528,486 published Mar. 4, 2003 (incorporated herein by reference and for all purposes). Specifically, exendin analogs include those consisting of an exendin or exendin analog having at least 90% homology to exendin-4 having optionally between one and five deletions at positions 34-39, and a C-terminal extension of a peptide sequence of 4-20 amino acid units covalently bound to said exendin wherein each amino acid unit in said peptide extension sequence is selected from the group consisting of Ala, Leu, Ser, Thr, Tyr, Asn, Gln, Asp, Glu, Lys, Arg, His, and Met. More preferably the extension is a peptide sequence of 4-20 amino acid residues, e.g., in the range of 4-15, more preferably in the range of 4-10 in particular in the range of 4-7 amino acid residues, e.g., of 4, 5, 6, 7, 8 or 10 amino acid residues, where 6 amino acid residues are preferred. Most preferably, according to U.S. Pat. No. 6,528,486 the extension peptide contains at least one Lys residue, and is even more preferably from 3 to 7 lysines and even most preferably 6 lysines.

For example, one analog is HGEGTFTSDLSKQMEEEAVRLFIEWLKNGGPSSG APPSKKKKKK (SEQ ID NO:41) (also designated ([des-³⁶Pro]exendin-4(1-39)-Lys₆). Additional exemplary analogs include Lys₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Lys)₆ (H-Lys₆-des Pro ³⁶exendin-4(1-39)-Lys₆) (SEQ ID NO:42); His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Trp-Leu-Lys-Asn-Gly-Gly-Pro-Ser-Ser-Gly-Ala-Ser (H-[des ^{36,37,38}Pro] exendin-4(1-39)-NH₂) (SEQ ID NO:43); Lys-Lys-Lys-Lys-Lys-Lys-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser (H-(Lys)₆-[des ^{36,37,38}Pro]exendin-4(1-39) (SEQ ID NO:44); Asn-Glu-Glu-Glu-Glu-Glu-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser (H-Asn-(Glu)₅-[des ^{36,37,38}Pro]exendin-4(1-39) (SEQ ID NO:45); His-Gly-Glu-Gly-Thr-Phe-Thr-Ser-Asp-Leu-Ser-Lys-Gln-Met-Glu-Glu-Glu-Ala-Val-Arg-Lue-Phe-Ile-Glu-Trp-Leu-Lys-Asn-Gly-Gly-Pro-Ser-Ser-Gly-Ala-Ser-(Lys)₆ ([des ^{36,37,38}Pro]exendin-4(1-39)-(Lys)₆) (SEQ ID NO:46); (Lys)₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ (H-(Lys)₆-[des ^{36,37,38}Pro]exendin-4(1-39)-(Lys)₆) (SEQ ID NO:47); and Asp-Glu-Glu-Glu-Glu-Glu-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ (Asn-(Glu)₅-[des ^{36,37,38}Pro]exendin-4(1-39)-(Lys)₆) (SEQ ID NO:48) As customary in the art, repetition of an amino acid can be indicated by a subscripted number setting forth the number of repetitions; i.e., Lys₆, (Lys)₆ and the like refer to hexalysyl. In any and each of the exendin analogs and formulas described above, specifically contemplated are analogs thereof further including a replacement for the histidine corresponding to position 1 made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl, dimethyl histidyl, imidazopropionyl and imidazoacetyl. Further specifically contemplated herein are exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl)carboxylic acid, or (1-aminocyclooctyl) carboxylic acid.

Further examples of exendin analogs suitable for use in the methods described herein are described in published PCT application WO2004035623 (incorporated herein by reference and for all purposes), particularly those which include naturally-occurring amino acids, which describes exendin analogs having at least one modified amino acid residue particularly at positions ¹³Gln, ¹⁴Met, ²⁵Trp or ²⁸Asn with reference to the corresponding positions of exendin-4(1-39). According to that publication are additional such analogs further including a 1-7 amino acid C-terminal extension that includes at least one lysine amino acid and more preferably at least five lysine amino acid units such as six or seven lysine amino acid units.

In any and each of the exendin analogs and formulas described above or herein, specifically contemplated are those wherein a replacement for the histidine corresponding to position 1 is made with any of L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine, 3-pyridylalanine, 2-pyridylalanine or 4-pyridylalanine, beta-hydroxyl-imidazopropionyl, dimethyl histidyl, imidazopropionyl, and imidazoacetyl. Particularly preferred are desaminohistidyl exendin-4, dimethyl histidyl exendin-4, beta-hydroxyl-imidazopropionyl exendin-4, imidazopropionyl exendin-4, and imidazoacetyl-exendin-4, and their Leu14 analogs. Further specifically contemplated herein are exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl) carboxylic acid, or (1-aminocyclooctyl) carboxylic acid.

Further examples of exendin analogs suitable for use in the methods described herein are those exendin containing hybrids described in published PCT applications WO2007022123, WO2005077072, and WO2011063414, incorporated herein by reference, including compounds which include the hybrids HGEGTFTSDLSKQLEEEAVRLFIEWLKNGGPSSGAPPPSGGG KCNTATCVLGRLSQELHRLQTYPRTNTGSNTY (SEQ ID NO:49) or its C-terminally amidated form HGEGTFTSDLSKQLEEEAVRLFIEWLKNGGPSSGAPPPSGGGKCNTAT CVLGRLSQELHRLQTYPRTNTGSNTY-NH2 (SEQ ID NO:50) or HGEGTFTSDLSKQLEEEAVRLFIEWLKQGGPSKEIISGGGKCNTATCVLGRLSQELHR LQTYPRTNTGSNTY (SEQ ID NO:51) and its C-terminally amidated form HGEGTFTSDLSKQ LEEEAVRLFIEWLKQGGPSKEIISGGGKCNTATCVLGRLSQELHRLQTYPRTNTGSNT Y-NH2 (SEQ ID NO:52).

Further examples of exendin analogs and derivatives that are long-acting compounds suitable for use in the methods described herein include long acting Fc acid albumin conjugates described in published United States Patent Application 20090238838, such as HM11260C, United States Patent Application 20090238838 discloses an exendin analog fused or conjugated to an immunoglobulin Fc region by a non-peptidyl polymer where the polymer is amongst other things a short polyethylene glycol and where one end of the non-peptidyl polymer is linked to an amino acid residue other than the N-terminus of the insulinotropic peptide, such as to the epsilon amino of Lys²⁷ in the exendin analog.

### III. Exemplary Syntheses

General methods of polypeptide synthesis. The polypeptide components of the compounds, e.g., the polypeptides having a covalently attached amine derivatizing agent described herein, may be prepared using biological, chemical, and/or recombinant DNA techniques known in the art. Exemplary methods includes those described herein and in U.S. Pat. No. 6,872,700; WO 2007/139941; WO 2007/140284; WO 2008/082274; WO 2009/011544; and US Publication No, 2007/0238669, the disclosures of which are incorporated herein by reference in their entireties and for all purposes.

For example, the polypeptide components may be prepared using standard solid-phase peptide synthesis techniques, such as an automated or semiautomated peptide synthesizer. Typically, using such techniques, an alpha-N-carbamoyl protected amino acid and an amino acid attached to the growing peptide chain on a resin are coupled at room temperature in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhdryl-amine resin used in the peptide synthesizer may be purchased from a variety of commercial sources, including for example Applied Biosystems Inc. (Foster City, Calif.).

For chemical synthesis solid phase peptide synthesis can be used for the polypeptide component of the disclosed compounds, since in general solid phase synthesis is a straightforward approach with excellent scalability to commercial scale, and is generally compatible with relatively long polypeptide conjugates. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See APPLIED BIOSYSTEMS USER's MANUAL for the ABI 430A PEPTITE SSYNTHIZER, Version 1.3B Jul. 1, 1988, section 6, pp. 43-70, Applied Biosystems, Inc., Foster City, Calif.) with capping. Boc-peptide-resins may be cleaved with HF (−5° C. to 0° C., 1 hour). The peptide may be extracted front the resin with alternating water and acetic acid, and the filtrates lyophilized. The Fmoc-peptide resins may be cleaved according to standard methods (e.g., INTRODUCTION to CLEAVAGE TECHNIQUES, Applied Biosystems, Inc., 1990, pp. 6-12). Peptides may be also be assembled using a variety of systems, including the Advanced Chem Tech Synthesizer (Model MPS 350, Louisville, Ky.).

Purification of compounds. Purification of compounds described herein generally follows methods available to the skilled artisan. In a typical purification procedure, a crude peptide is initially purified via ion exchange chromatography, e.g., Macro Cap SP cation exchanger column. A typical purification procedure employs Buffer A (20 mM sodium acetate buffer, pH 5.0) and Buffer B (20 mM sodium acetate buffer, pH 5.0, 0.5 M sodium chloride) in a gradient elation program, e.g., 0-0% Buffer B (20 min), followed by 0-50% Buffer B (50 min), then 100% Buffer B (20 min). The flow rate is typically 3 mL/min. SDS (i.e., sodium dodecylsulfate) polyacrylamide gel visualization of the collected fractions is conducted, followed by dialysis against water of the suitable fraction pool and lyophilization of the resultant. Analytical characterization typically employs MALDI mass spectroscopy.

### IV. Assays

The compounds described herein may be tested alone or in combination according to embodiments described herein in a variety of assays (e.g., receptor binding assays) using methodologies generally known to those skilled in the art. Such assays include those described herein. Methods for production and assay of compounds described herein are generally available to the skilled artisan. Further, specific methods are described herein as well as in the patent publications and other references cited herein, which are incorporated by reference for this and all purposes.

Blood glucose. Blood glucose can be measured by any of a variety of commercially available test kits, e.g., OneTouch® Ultra® (LifeScan, Inc. Milpitas, Calif.). Peptide can be injected IP at zero time immediately followed by baseline sampling in 2-hr lasted NIH/Swiss mice.

GLP-1 receptor binding and functional assays: GLP-1 receptor binding activity and affinity may be measured in any number of known methods. For example, in one method binding activity is measured using a binding displacement assay in which the receptor source is RINm5F cell membranes, and the ligand is [¹²⁵I]GLP-1 or iodinated exendin(1-39) or iodinated exendin(9-39). Homogenized RINm5F cell membranes are incubated in 20 mM HEPES buffer with 40,000 cpm [¹²⁵I]GLP-1 (or exendin) tracer, and varying concentrations of test compound for 2 hours at 23° C. with constant mixing. Reaction mixtures are filtered through glass filter pads presoaked with 0.3% PEI solution and rinsed with ice-cold phosphate buffered saline. Bound counts are determined using a scintillation counter. Binding affinities can be calculated using GraphPad Prism® software (GraphPad Software, Inc., San Diego, Calif.).

In vitro assays for functional GLP-1 receptor activation can be performed using known methods and cells and tissues. For example, exendin-4 stimulation of GLP-1 receptor bearing cells can induce an increase in adenylate cyclase activation, cAMP synthesis, membrane depolarization, rise in intracellular calcium and increase in glucose-induced insulin secretion (Holz et al, 1995, J. Biol. Chem. 270(30):17749-57). Assays may be performed with-or without the presence of albumin.

In vivo assays for activity and duration of action and pharmacokinetics can be done using known methods. For example, duration can be performed using an oral glucose tolerance test (OGTT) in which the drug is administered to the subject at a desired time point before the glucose is administered orally (to measure drug duration of action; OGTT DOA) and glucose blood levels are measured (e.g. readily done in mice). Activity and duration can also be measured using an intravenous glucose tolerance test (IVGTT) in which the drug is administered to the subject at a desired time point before the glucose is administered IV (IVGTT DOA) and blood glucose levels are measured (e.g. can readily be done in rats). Preferred compounds have a desired effect on blood glucose of at least 24 hours duration after a single dose of drug, preferably at least 3 days, at least 4 days, at least 5 days and at least 1 week alter the single dose of drug is given.

### V. Methods of Use

In a first aspect, there is provided a method of covalently attaching an amine derivatizing agent to a glutamine of a first polypeptide. The method includes adding an amine derivatizing agent, a transglutaminase, a first polypeptide including a glutamine residue, and a co-solvent to a reaction mixture. The method further includes allowing the amine derivatizing agent to react with the glutamine residue in the reaction mixture to form an amide bond, thereby covalently attaching the amine derivatizing agent to the glutamine residue of the first polypeptide.

The term “amine derivatizing agent” refers to compounds and derivatives and analogs thereof having a free amine group suitable for reaction with the side chain of a glutamine residue of a polypeptide, e.g., a first polypeptide as disclosed herein, in the presence of a transglutaminase to form an amide bond, thereby covalently attaching the amine derivatizing agent to the glutamine of the polypeptide. Exemplary amine derivatizing agents include imaging label amine derivatizing agents, fluorescent label amine derivatizing agents, fatty acid amine derivatizing agents, bile acid amine derivatizing agents, glycan amine derivatizing agents, nasal delivery enhancing moiety amine derivatizing agents, biotin amine derivatizing agents, cobalamine amine derivatizing agents or derivatives thereof, amphiphilic oligomer amine derivatizing agents, water soluble polymer amine derivatizing agents, and polypeptide amine derivatizing agents, as disclosed herein.

A water soluble polymer amine derivatizing agent is an amine deriviatizing agent including a water soluble polymer. The term “water soluble polymer” means a polymer which is sufficiently soluble in water under physiologic conditions of e.g., temperature, ionic concentration and the like, as known in the art, to be useful for the methods and compounds described herein. A wafer soluble polymer can increase the solubility of a peptide or other biomolecule to which such water soluble polymer is attached. Indeed, such attachment has been proposed as a means for improving the circulating life, water solubility and/or antigenicity of administered proteins in vivo. See e.g., U.S. Pat. No. 4,179,337; U.S. Published Appl.No. 2008/0032408. Many different water-soluble polymers and attachment chemistries have been used towards this goal, such as polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and the like. Additional exemplary water-soluble polymers include hydroxyalkylcellulose (e.g., hydroxymethylcellulose, hydroxyethylcellulose, and the like), latex powders, alkylcellulose (e.g., ethylcellulose) polymers, cellulose ethers, polyethers, poly(acrylamide/acrylic acid), dextrans, and the like.

In one embodiment, linkers or amine derivatizing agents contemplated herein include a polyethylene glycol (“polyethylene glycol amine derivatizing agent”). Polyethylene glycol (“PEG”) has been used in efforts to obtain therapeutically usable polypeptides. See e.g., Zalipsky, S., 1995, Bioconjugate Chemistry, 6; 150-165; Mehvar, R., 2000, J. Pharm. Pharmaceut. Sci., 3:125-136. As appreciated by one of skill in the art, the PEG backbone [(CH₂CH₂—O—)ₙ, n: number of repeating monomers] is flexible and amphiphilic. Without wishing to be bound by any theory or mechanism of action, the long, chain-like PEG molecule or moiety is believed to be heavily hydrated and in rapid motion when in an aqueous medium. This rapid motion is believed to cause the PEG to sweep out a large volume and prevents the approach and interference of other molecules. As a result, when attached to another chemical entity (such as a polypeptide), PEG polymer chains can protect such chemical entity from immune response and other clearance mechanisms. As a result, pegylation (i.e., covalent addition of PEG) can lead to improved drug efficacy and safety by optimizing pharmacokinetics, increasing bioavailability, and decreasing immunogenicity and dosing frequency. “Pegylation” refers in the customary sense to conjugation (i.e., chemical bonding) of a PEG moiety with another compound. For example, attachment of PEG has been shown to protect proteins against proteolysis. See e.g., Blomhoff, H. K. et al., 1983, Biochim Biophys Acta, 757:202-208. Unless expressly indicated to the contrary, the terms “PEG,” “polyethylene glycol polymer” and the like refer to polyethylene glycol polymer and derivatives thereof, including methoxy-PEG (mPEG).

A variety of means have been used to attach polymer moieties such as PEG and related polymers to reactive groups found on the protein. See e.g., U.S. Pat. No. 4,179,337; U.S. Pat. No. 4,002,531; Abuchowski et al., 1981, in “Enzymes as Drugs,” J. S. Holcerberg and J. Roberts, (Eds.), pp. 367-383; Zalipsky, S., 1995, Bioconjugate Chemistry, 6:150-165. The use of PEG and other polymers to modify proteins has been discussed. See e.g., Cheng, T.-L. et al., 1999, Bioconjugate Chem., 10:520-528; Belcheva, N. et al., 1999, Bioconjugate Chem., 10:932-937; Bettinger, T. et al., 1998, Bioconjugate Chem., 9:842-846; Huang, S.-Y, et al., 1998, Bioconjugate Chem., 9:612-617; Xu, B. et al. 1998, Langmuir, 13:2447-2456; Schwarz, J. B. et al., 1999, J. Amer. Chem. Soc., 121:2662-2673; Reuter, J. D. et al., 1999, Bioconjugate Chem., 10:271-278; Chan, T.-H. et al., 1997, J. Org. Chem., 62:3500-3504. Typical attachment sites in proteins include primary amino groups, such as those on lysine residues or at the N-terminus, thiol groups, such as those on cysteine side-chains, and carboxyl groups, such as those on glutamate or aspartate residues or at the C-terminus. Common sites of attachment are to the sugar residues of glycoproteins, cysteines or to the N-terminus and lysines of the target polypeptide.

In some embodiments, a PEG moiety in a polypeptide conjugate described herein has a nominal molecular weight within a specified range. As customary in the art, the size of a PEG moiety is indicated by reference to the nominal molecular weight, typically provided in kilodaltons (kDa). The molecular weight is calculated in a variety of ways known in the art, including number, weight, viscosity and “Z” average molecular weight. It is understood that polymers, such as PEG and the like, exist as a distribution of molecule weights about a nominal average value.

Exemplary of the terminology for molecular weight for PEGs, the term “mPEG40KD” refers to a methoxy polyethylene glycol polymer having a nominal molecular weight of 40 kilodaltons. Reference to PEGs of other molecular weights follows this convention. In some embodiments, the PEG moiety has a nominal molecular weight in the range 10-100 kDa, 20-80 kDa, 20-60 kDa, or 20-40 kDa. In some embodiments, the PEG moiety has a nominal molecular weight of 10, 15, 20, 25, 30, 35, 40, 45, 30, 55, 60, 65, 70, 75, 80, 85, 90, 95 or even 100 kDa. Preferably, the PEG moiety has a molecular weight of 20, 25, 30, 40, 60 of 80 kDa. In one embodiment, the PEG is monodisperse as known in the art. In the nomenclature of monodisperse PEG species the term “d” (i.e., “discrete”) is typically appended to the name of the PEG. For example, the term “m-dPEG24” refers to discrete methoxy PEG having 24 ethyleneglycol monomers.

PEG molecules useful for derivatization of polypeptides are typically classified into linear, branched and Warwick (i.e., PolyPEG®) classes of PEGs, as known in the art. Unless expressly indicated to the contrary, the PEG moieties described herein are linear PEGs. Furthermore, the terms “two arm branched,” “Y-shaped” and the like refer to branched PEG moieties, as known in the art. The term “Warwick” in the context of PEGs, also known as “comb” or “comb-type” PEGs, refers to a variety of multi-arm PEGs attached to a backbone, typically poly(methacrylate), as known in the art.

The terms “first polypeptide” and the like refer to polypeptides disclosed herein including amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog or fragment thereof. In one embodiment, the first polypeptide includes a linker, which linker includes a glutamine residue available for reaction with the transglutaminase in the methods disclosed herein.

Accordingly, the terms “first polypeptide comprising a glutamine” and the like refer to a first polypeptide having a glutamine residue side chain available for reaction with an amine derivatizing agent in the presence of a transglutaminase, e.g., in a reaction mixture further including a co-solvent. In one embodiment, the glutamine residue is a naturally occurring residue in the sequence of the first polypeptide. In one embodiment, the glutamine residue resulting from a substitution or insertion in an analog of the first polypeptide. In one embodiment, the first polypeptide includes a linker, as described herein, which linker includes a glutamine residue.

The term “transglutaminase” refers in the customary sense to a polypeptide having enzymatic activity to catalyze the aminolysis of the γ-carboxamide group of the glutamine side chains of a substrate, e.g., a peptide substrate. A typical reaction is disclosed in Scheme 1 following, wherein R—CONH₂ represents the acceptor, and R′—NH₂ is the donor amine, e.g., alkylamine.

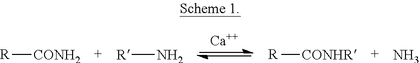

The reaction proceeds via an acyl-transfer mechanism in which the γ-carboxamide group acts as an acyl donor and suitably unbranched primary amines act as acyl acceptors. Accordingly, the reaction catalyzed by transglutaminase offers a method for selective introduction of functional groups into proteins under mild conditions. See e.g., Coussons et al., 1992, Biochem J. 283:803-806.

The term “co-solvent”as used herein refers to a compound or plurality of compounds included with a primary solvent, e.g., water, in a reaction buffer useful in the methods described herein. Under the homogeneous aqueous conditions contemplated herein, the primary solvent is water. The term “reaction buffer” refers to a homogeneous composition forming the liquid phase of a reaction mixture which is suitable for a transglutaminase to affect an acyl-transfer reaction. The term “reaction mixture” refers to a reaction buffer which further includes amine derivatizing agent, first polypeptide including a glutamine, and transglutaminase of the methods disclosed herein. The reaction buffer or reaction mixture can additionally include buffers (e.g., pH or redox buffers as known in the art), metal ions (e.g., Ca⁺⁺, Mg⁺⁺, CT, and the like), or cofactors required for activity of the transglutaminase, as known in the art.

In one embodiment, the co-solvent is an organic compound. In one embodiment, the co-solvent is a first water-soluble polymer. In one embodiment, the co-solvent is a polyether, e.g., polyethylene glycol, polypropylene glycol, and the like. In one embodiment, the co-solvent is a polysorbate, as known in the art. In one embodiment, the co-solvent is a solid at room temperature prior to dissolution in the reaction buffer or reaction mixture. In one embodiment, the co-solvent is a liquid at room temperature. In one embodiment, the mixture of the primary solvent, optional buffers, metal ions and cofactors, and co-solvent affords a homogenous liquid milieu which is suitable for a transglutaminase to affect an acyl-transfer reaction upon addition of an amine derivatizing agent, a first polypeptide including a glutamine, and a transglutaminase disclosed herein.

In one embodiment, co-solvent is present in the reaction mixture in a range from about 0.1% to about 50%, about 0.1% to about 40%, about 0.1% to about 30%, about 0.1% to about 20%, about 0.1% to about 10%, about 0.1% to about 5%, about 0.1% to about 4%, about 0.1% to about 3%, about 0.1% to about 2%, or about 0.1% to about 1%. In one embodiment, the co-solvent is present at a concentration of about 50%, 45%, 40%, 35%, 30%, 28%, 26%, 24%, 22%, 20%, 19%, 18%, 17%, 16%, 15%, 14%, 13%, 12%, 11%, 10%, 9%, 8%, 7%, 6%, 5%, 4%, 3%, 2%, or 1%. In one embodiment, co-solvent is present in the reaction mixture at 20%. In one embodiment, co-solvent is present in the reaction mixture at 15%. In one embodiment, co-solvent is present in the reaction mixture at 10%. In one embodiment, co-solvent is present in the reaction mixture at 5%. In one embodiment, the reaction mixture includes at least 50% of the co-solvent. In one embodiment, the reaction mixture includes at least 40% of the co-solvent. In one embodiment, the reaction mixture includes at least 30% of the co-solvent. In one embodiment, the reaction mixture includes at least 20% of the co-solvent. In one embodiment, the reaction mixture includes at least 10% of the co-solvent. In one embodiment, the reaction mixture includes at least 9% of the co-solvent. In one embodiment, the reaction mixture includes at least 8% of the co-solvent. In one embodiment, the reaction mixture includes at least 8% of the co-solvent. In one embodiment, the reaction mixture includes at least 6% of the co-solvent. In one embodiment, the reaction mixture includes at least 5% of the co-solvent. In one embodiment, the reaction mixture includes at least 4% of the co-solvent. In one embodiment, the reaction mixture includes at least 3% of the co-solvent. In one embodiment, the reaction mixture includes at least 2% of the co-solvent. In one embodiment, the reaction mixture includes at least 1% of the co-solvent. Absent express indication otherwise, the term “%” in the context of a concentration refers, in the customary sense, to weight percentage (i.e., “w/w”). Absent express indication otherwise, the term “about” in the context of a numeric value refers to the nominal value ±10% thereof.

As understood in the art, the reaction catalyzed by a transglutaminase may afford a mixture of reactants and products at equilibrium. See Scheme 1. Accordingly, the reaction of the amine derivatizing agent with the glutamine of the first polypeptide disclosed herein may achieve an extent of conversion less than 100% due to the equilibrium constant characterizing the aminolysis of the γ-carboxamide group of the glutamine side chains of the peptide substrate. Moreover, substrates and products of the transglutaminase reaction may undergo additional reactions, e.g., peptide bond scission, reaction at another glutamine, and the like, which can also decreased the extent of reaction of the chemical mechanism depicted in Scheme 1. The terms “side reaction products” and the like in the context of the transglutaminase catalyzed reactions described herein refer to products resulting from additional reactions described herein.

In has been found, surprisingly, that incorporation of a co-solvent into a reaction mixture as described herein can facilitate the formation of product in the transglutaminase catalyzed aminolysis of the γ-carboxamide group of the glutamine side chains of peptide substrates. Specifically, in one embodiment, the extent of covalently attaching an amine derivatizing agent to a glutamine of a first polypeptide is at least 50%, e.g., 50%, 60%, 70%, 80%, 90%, 92%, 94%, 96%, 98%, 99% or even greater. The terms “extent of covalently attaching” and the like refer to the extent of the reaction of the transglutaminase disclosed herein. In one embodiment, the extent of covalently attaching an amine derivatizing agent to a glutamine of a first polypeptide is at least 50%. In one embodiment, the extent of covalently attaching an amine derivatizing agent to a glutamine of a first polypeptide is at least 60%. In one embodiment, the extent of covalently attaching an amine derivatizing agent to a glutamine of a first polypeptide is at least 70%. In one embodiment, the extent of covalently attaching an amine derivatizing agent to a glutamine of a first polypeptide is at least 80%. In one-embodiment, the extent of covalently attaching an amine derivatizing agent to a glutamine of a first polypeptide is at least 90%. In one embodiment, the extent of covalently attaching an amine derivatizing agent to a glutamine of a first polypeptide is greater than 90%, e.g., 92%, 94%, 96%, 98%, 99%, or even greater.

In one embodiment, the co-solvent is a glycol. In one embodiment, the glycol is propylene glycol or polyethylene glycol. In one embodiment, the glycol is propylene glycol. In one embodiment, the glycol is polyethylene glycol. In one embodiment, the glycol is propylene glycol or polyethylene glycol present at at least 1% (w/w), e.g., 1%, 2%, 3%, 4%, 5%, 10%, 15%, 20%, 25%, 30%, 35%, 40%, or even 50%. In one embodiment, the glycol is propylene glycol present at at least 1% (w/w), e.g., 1%, 2%, 3%, 4%, 5%, 10%, 15%, 20%, 25%, 30%, 35%, 40%, or even 50%. In one embodiment, the glycol is polyethylene glycol present at at least 1% (w/w), e.g., 1%, 2%, 3%, 4%, 5%, 10%, 15%, 20%, 25%, 30%, 35%, 40%, or even 50%.

In one embodiment of the method disclosed above wherein co-solvent is present in the reaction mixture, a lower concentration of side reaction products obtains relative to the corresponding method wherein the co-solvent is absent from the reaction mixture. In one embodiment, the lower concentration of side reaction products is 25% or less, e.g., 25%, 20%, 15%, 10%, 5%, or even less relative to the corresponding method wherein the co-solvent is absent from the reaction mixture.

Further to any of the method disclosed above, in one embodiment the first polypeptide includes a peptide hormone, or analog, derivative or fragment thereof. In one embodiment, the first polypeptide is a peptide hormone. In one embodiment, the first polypeptide is an analog of a peptide hormone. In one embodiment, the first polypeptide is a derivative of a peptide hormone. In one embodiment, the first polypeptide is a fragment of a peptide hormone. In one embodiment, the first peptide, when covalently attached to the amine derivatizing agent, exhibits at least one hormonal activity. The term “hormonal activity” refers in the customary sense to activity which can elicit a biological response, as judged by assays known in the art.

In one embodiment, the first polypeptide includes an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog, derivative or fragment thereof. In one embodiment, the first polypeptide includes amylin, pramlintide, adrenomedullin (ADM), calcitonin (CT), calcitonin gene related peptide (CGRP), intermedin, cholecystokinin (CCK), leptin, peptide YY (PYY), glucagon-like peptide-1 (GLP-1), glucagon-like peptide 2 (GLP-2), oxyntomodulin (OXM), natriuretic peptide, urocortin, neuromedin family peptide, exendin, or analog or fragment thereof.

In one embodiment, the first polypeptide further includes a linker covalently attached thereto. In one embodiment, the linker is attached to a non-peptidic moiety. In one embodiment, the linker is attached to a glutamine residue. In one embodiment, the linker includes a glutamine residue having an amide side chain functionality suitable for use in the methods disclosed herein.

In one embodiment, the first polypeptide includes an exendin, or analog, derivative, or fragment thereof. In one embodiment, the first polypeptide includes an exendin analog or fragment thereof. In one embodiment, the exendin analog has at least 80% sequence identity, e.g. 80%, 82%, 84%, 86%, 88%, 90%, 92%, 94%, 96%, or even greater sequence identity, with exendin-4. In one embodiment, the exendin analog has at least 80% sequence identity with exendin-4. In one embodiment, the exendin analog has at least 90% sequence identity with exendin-4. In one embodiment, the exendin is exendin-3 or exendin-4. In one embodiment, the exendin is exendin-3. In one embodiment, the exendin is exendin-4.

Further to the method disclosed herein having a defined first polypeptide, in one embodiment the amine derivatizing agent includes an imaging label, a fluorescent label, a fatty acid, a bile acid, a glycan, a nasal delivery enhancing compound, biotin, cobalamine or derivative thereof, an amphiphilic oligomer, a second water soluble polymer, or a second polypeptide. In one embodiment, the amine derivatizing agent includes an imaging label. In one embodiment, the amine derivatizing agent includes an fluorescent label. In one embodiment, the amine derivatizing agent includes a fatty acid. In one embodiment, the amine derivatizing agent includes a bile acid. In one embodiment, the amine derivatizing agent includes cholic acid. In one embodiment, the amine derivatizing agent includes a glycan. In one embodiment, the amine derivatizing agent includes a nasal delivery enhancing compound. In one embodiment, the amine derivatizing agent includes biotin. In one embodiment, the amine derivatizing agent includes cobalamine or derivative thereof. In one embodiment, the amine derivatizing agent includes an amphiphilic oligomer. In one embodiment, the amine derivatizing agent includes a second water-soluble polymer In one embodiments, the second water-soluble polymer is polyethylene glycol.

The term “imaging label” refers in the customary sense to a composition or functionality useful for imaging. Methods of imaging include inter alia nuclear magnetic resonance (NMR), electron paramagnetic spin (EPR), spectroscopy (e.g., UV-visible or IR spectroscopy), electron microscopy (e.g., conjugates including ferritin conjugates suitable for direct visualization via electron microscopy), fluorescence, positron-electron tomography (PET), and the like. The term “fluorescent label” refers, as customary in the art, to a compound or functionality which is a fluorophore. The term “fatty acid” refers, as customary in the art, to a carboxylic acid having a aliphatic tail (e.g., C₆-C₂₄), which is saturated or unsaturated, which tail can be unsubstituted or substituted as disclosed herein. The term “bile acid” refers, in the customary sense, to steroid acids found predominantly in the bile of mammals. The term “a glycan” refers, in the customary sense, to a polysaccharide or oligosaccharide, which can be heteropolymers or homopolymers of monosaccharide residues.

The term “a nasal delivery enhancing compound” refers, in the customary sense, to a composition or functionality which facilitates increased intranasal bioavailability of peptide and protein drugs. Exemplary nasal delivery enhancing, compounds include, inter alia, substituted and unsubstituted cyclodextrins, Zot peptides, and PAR-2 agonists. The terms “Zot peptide” and the like refer to the zonula occludens toxin found, e.g., in Vibrio cholerae, which reversibly regulates tight junction permeability. See e.g., Wang, W., et al., 2000, J. Cell Science 113:4435-4440; Fasano, A., et al., 1991, Proc. Natl. Acad. Sci. USA 88:5242-3246; Baudry, B. et al., 1992, Immun. 60: 428-434. The sequence of Zot from V. chaterae BX 330286 follows: MSIFIHHGAPGSYKTSGALWLRLLPAIKSGRHIITNVRGLNLERMAKYLKMDVSDISI EFIDTDHPDGRLTMARFWHWARKDAFLFIDECGRIWPPRLTATNLKALDTPPDLVAE DRPESFEVAFDMHRHHGWDICLTTPNIAKVHNMIREAAEIGYRHFNRATVGLGAKFT LTTHDAANSGQMDSHALTRQVKKIPSPIFKMYASTTTGKARDTMAGTALWKDRKIL FLFGMVFLMFSYSFYGLHDNPIFTGGNDATIESEQSEPQSKATAGNAVGSKAVAPAS FGFCIGRLCVQDGFVTVGDERYRLVDNFDIPYRGLWATGHHIYKDTLTVFFETESGS VPTELFASSYRYKVLPLPDFNHFVVFDTFTAQALWVEVKRGLPIKTENDKKGLNSIF (SEQ ID NO:53). The biologically active portion of the toxin has been mapped to residues 288-293 with sequence FCIGRL (SEQ ID NO:54). See e.g., Di Pierro, M., et al., 2001, J. Biol. Chem. 276:19160-19165. Accordingly, in one embodiment the amine-derivatizing agent includes an analog, derivative or fragment of a Zot peptide. In one embodiment the amine-derivatizing agent includes an analog, derivative or fragment of a biologically active portion of Zot peptide.

The term “cyclodextrin” refers in the customary sense to the family of cyclic polysaccharides which include α-D-glucopyranoside units linked via 1→4 linkage, as known in the art. Cyclodextrins are useful to improve the nasal absorption of drugs with low oral bioavailability. Accordingly, in one embodiment, the amine-derivatizing agent includes a cyclodextrin or derivative thereof. The term “PAR-2 agonist” refers in the customary sense to agonists for protein-activated receptor-2 (PAR-2). The term “amphiphilic oligomer” refers in the customary sense to an oligomer having both hydrophilic and hydrophobic properties. An exemplary amphiphilic oligomer is polyethylene glycol. Further exemplary amphiphilic oligomers includes polyamides, polyesters, polyureas, polycarbonates, polyurethanes, polyphenylenes and heteroarylene polymers, as known in the art.

In one embodiment, the method provides a compound which includes a first polypeptide covalently attached to an amine derivatizing agent, the compound having the components of any one of compound embodiments 1-176 as set forth in Table 1 following. In particular, in one embodiment any one of first polypeptides A-P as set forth in Table 1 (see e.g., legend following) is in combination with any one of amine derivatizing agents a-k as set forth in Table 1.

TABLE 1
Expemplary Compound Embodiments

| | | First polypeptide | | | | | | | | | | | | | | | |
|---|---|---|---|---|---|---|---|---|---|---|---|---|---|---|---|---|---|
| | | A | B | C | D | E | F | G | H | I | J | K | L | M | N | O | P |
| Amine | a | 1 | 2 | 3 | 4 | 5 | 6 | 7 | 8 | 9 | 10 | 11 | 12 | 13 | 14 | 15 | 16 |
| Derivatizing | b | 17 | 18 | 19 | 20 | 21 | 22 | 23 | 24 | 25 | 26 | 27 | 28 | 29 | 30 | 31 | 32 |
| Agent | c | 33 | 34 | 35 | 36 | 37 | 38 | 39 | 40 | 41 | 42 | 43 | 44 | 45 | 46 | 47 | 48 |
| | d | 49 | 50 | 51 | 52 | 53 | 54 | 55 | 56 | 57 | 58 | 59 | 60 | 61 | 62 | 63 | 64 |
| | e | 65 | 66 | 67 | 68 | 69 | 70 | 71 | 72 | 73 | 74 | 75 | 76 | 77 | 78 | 79 | 80 |
| | f | 81 | 82 | 83 | 84 | 85 | 86 | 87 | 88 | 89 | 90 | 91 | 92 | 93 | 94 | 95 | 96 |
| | g | 97 | 98 | 99 | 100 | 101 | 102 | 103 | 104 | 105 | 106 | 107 | 108 | 109 | 110 | 111 | 112 |
| | h | 113 | 114 | 115 | 116 | 117 | 118 | 119 | 120 | 121 | 122 | 123 | 124 | 125 | 126 | 127 | 128 |
| | i | 129 | 130 | 131 | 132 | 133 | 134 | 135 | 136 | 137 | 138 | 139 | 140 | 141 | 142 | 143 | 144 |
| | j | 145 | 146 | 147 | 148 | 149 | 150 | 151 | 152 | 153 | 154 | 155 | 156 | 157 | 158 | 159 | 160 |
| | k | 161 | 162 | 163 | 164 | 165 | 166 | 167 | 168 | 169 | 170 | 171 | 172 | 173 | 174 | 175 | 176 |

Legend: First polypeptide: A: amylin, or analog or fragment thereof; B: pramlintide, or analog or fragment thereof; C: adrenomedullin (ADM), or analog or fragment thereof; D: calcitonin (CT), or analog or fragment thereof; E: calcitonin gene related peptide (CGRP), or analog or fragment thereof; F: intermedin, or analog or fragment thereof; G: a cholecystokinin (CCK), or analog or fragment thereof; H: a leptin, or analog or fragment thereof; I: peptide YY (PYY), or analog or fragment thereof; J: glucagon-like peptide-1 (GLP-1), or analog or fragment thereof; K: glucagon-like peptide 2 (GLP-2), or analog or fragment thereof; L: oxyntomodulin (OXM), or analog or fragment thereof; M: a natriuretic peptide, or analog or fragment thereof; N: a urocortin, or analog or fragment thereof; O: a neuromedin family peptide, or analog or fragment thereof; P: an exendin, or analog or fragment thereof. Amine derivatizing agent: a: an imaging label; b: a fluorescent label; c: a fatty acid; d: a bile acid; e: a glycan; f: a nasal delivery enhancing compound; g: biotin; h: cobalamine or derivative thereof; i: an amphophilic oligomer; j: a second water soluble polymer; k: a second polypeptide.

In one embodiment, the compound embodiment set forth in Table 1 includes a first polypeptide (i.e., first polypeptide A-P) which is an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, or an exendin, or an analog, derivative or fragment thereof.

In one embodiment, the amine derivatizing agent to Table 1 is an imaging label amine derivatizing agent, a fluorescent label amine derivatizing agent, a fatty acid amine derivatizing agent, a bile acid amine derivatizing agent, a glycan amine derivatizing agent, a nasal delivery enhancing moiety amine derivatizing agent, a biotin amine derivatizing agent, a cobalamine amine derivatizing agent or derivative thereof, an amphiphilic oligomer amine derivatizing agent, a second water soluble polymer amine derivatizing agent, or a second polypeptide amine derivatizing agent.

In one embodiment, the amine derivatizing agent is an imaging label amine derivatizing agent. See Table 1, entry “a.” An imaging label amine derivatizing agent is an amine derivatizing agent including an imaging label moiety and an amine moiety. Exemplary imaging label moieties are known in the art and include chemiluminescent moieties, bioluminescent moieties, and fluorescent moieties. Further exemplary imaging label moieties include metal chelation moieties suitable for chelating metals including radionuclides. Metal chelation moieties include moieties having a plurality of functional groups, e.g., thiol, carboxyl, imidazoyl, and the like) with sufficient proximity and geometrical positioning to chelate a metal, including protoporphyrin derivatives, polypeptides (e.g., “Zinc finger” polypeptides, polyhistidine, and the like as known in the art), and polycarboxylic acid containing moieties. For example, exemplary metal chelation moieties further include moieties having a monovalent form of 1,4,7,10-tetraazacyclododecase-1,4,7,10-tetraacetic acid (DOTA), ethylenediaminetetraacetic acid (EDTA), diethylenetriaminepentaacetic acid (DTPA), and the like which have been derivatized as necessary to include a primary or second amine functionality. See e.g., Li, M. & Selvin, P. R., 1997, Bioconjugate Chem. 8:127-132. Chelation of a suitable metal or radionuclide, e.g., Gd, Ga, In, Tb, Tc, and the like can provide a species useful for imaging via NMR, EPR, positron-electron tomography, and UV-visible spectrometry, as known in the art. Methods of attaching a primary or second amine are well known in the art, and include reaction with bifunctional reagents, e.g., alkyldiamine, aminomethylphenol, lysine and higher and lower order homologs thereof, and the like, wherein the pendant amine becomes available to participate in the transglutaminase catalyzed acyl transfer reaction of the methods disclosed herein. Moreover, reaction of a bifunctional reagent with ferritin to provide a primary or second amine is useful for the synthesis of reagents useful for electron microscopy, as known in the art.

In one embodiment, the amine derivatizing agent is a fluorescent label amine derivatizing agent. See Table 1, entry “b.” A fluorescent label amine derivatizing agent is an amine derivatizing agent including a fluorescent moiety and an amine moiety. Fluorescent label amine derivatizing agents are commercially available, and can be synthesized by methods well known in the art. An exemplary fluorescent label amine derivatizing agent is dansyl cadaverine. See Example 8. Other exemplary fluorescent label amine derivatizing agents employ longer or shorter diamines in place of cadaverine, e.g., C₄-C₁₆ diamines. In one embodiment, the fluorescent label amine derivatizing agent is dansyl cadaverine.

In one embodiment, the amine derivatizing agent is a fatty acid amine derivatizing agent. See Table 1, entry “c.” A fatty acid amine derivatizing agent is an amine derivatizing agent including a fatty acid moiety and an amine moiety. Exemplary fatty acid amine derivatizing agents include lysine, or homolog thereof, wherein the alpha-amino functionality of the lysine or homolog thereof forms an amide bond with the carboxylate functionality of a fatty acid. See e.g., Example 4. In one embodiment, the fatty acid moiety includes a C₄-C₂₄ alkyl. In one embodiment, the fatty acid amine derivatizing agent is N_{α}-octan-1-amido lysine.

In one embodiment, the amine derivatizing agent is a bile acid amine derivatizing agent. See Table 1, entry “d.” A bile acid amine derivatizing agent is an amine derivatizing agent including a bile acid moiety and an amine moiety. An exemplary bile acid amine derivatizing agent is obtained by the formation of a cholic acid bonded to an oligopeptide (e.g., a dipeptide such as glycyllysine), wherein the terminal amino acid residue of the oligopeptide is a lysine or homolog thereof which provides a pendant amine functionality. The pendant amine functionality of the terminal residue provides the amine substrate for the transglutaminase reaction. See Example 5.

In one embodiment, the amine derivatizing agent is a glycan amine derivatizing agent. See Table 1, entry “e.” A glycan amine derivatizing agent is an amine derivatizing agent including a glycan moiety and an amine moiety. As known in the art, glycan moieties linked at nitrogen (i.e., “N-linked glycans”) include N-acetyl galactosamine, glucose, galactose, neuraminic acid, N-acetylglucosamine, fructose, mannose, fucose and other monosaccharides. See e.g., U.S. Pat. No. 8,137,954. Similarly, glycan moieties linked at oxygen (i.e., “O-linked glycans”) are well known in the art. See e.g., U.S. Pat. No. 8,063,015. Indeed, a variety of glycans having reactive primary amine at the reducing terminal are available commercially, as known in the art. In one embodiment, the glycan amine derivatizing agent is a dextran amine.

In one embodiment, the amine derivatizing agent is a nasal delivery enhancing moiety amine derivatizing agent. See Table 1, entry “f.” A nasal delivery enhancing moiety amine derivatizing agent is an amine derivatizing agent including a nasal delivery enhancing moiety and an amine moiety. Exemplary nasal delivery enhancing compounds include, inter alia, substituted and unsubstituted cyclodextrins, Zot peptides, and PAR-2 agonists. In one embodiment, the nasal delivery enhancing moiety includes a Zot peptide or analog thereof. In one embodiment, the nasal delivery enhancing moiety includes FXIGRLK (SEQ ID NO:55), wherein “X” is a surrogate for cysteine. Exemplary cysteine surrogates includes alanine, penicillamine, allylglycine, and the like. In one embodiment, the nasal delivery enhancing moiety amine derivatizing agent is FXIGRLK-amide (SEQ ID NO:56), where X is allylglycine. See Example 11.

In one embodiment, the amine derivatizing agent is a biotin amine derivatizing agent. See Table 1, entry “g.” A biotin amine derivatizing agent is an amine derivatizing agent including a biotin moiety and an amine moiety. As well known in the art, the carboxylate functionality of biotin can react with a diamine, e.g., an alkyl diamine, to form an amide bond and a pendant amine. The pendant amine can then function as a substrate in the transglutaminase reaction disclosed herein. In one embodiment, the biotin containing amine derivatizing agent is a biotin cadaverine amide, wherein cadaverine forms an amide linkage with the carboxylate of biotin, and the pendant amine is available as a substrate for the transglutaminase reaction. See Example 6.

In one embodiment, the amine derivatizing agent is a cobalamine amine derivatizing agent or derivative thereof. See Table 1, entry “h.” A cobalamine amine derivatizing agent is an amine derivatizing agent including a cobalamine moiety or derivative thereof and an amine moiety. In one embodiment, cyanocobalamine is bonded at the 5′ hydroxyl of the deoxyribofuranosyl ring of the deoxyadenosyl moiety with alkyldiamine, e.g., hexane-1,6-diamine, in a carbamate linkage. Methods to afford such carbamates are well known in the art, including the Curtius Rearrangement wherein isocyanate reacts with the 5′-hydroxyl to form the carbamate. See Example 7.

In one embodiment, the amine derivatizing agent is an amphiphilic oligomer amine derivatizing agent. See Table 1, entry “i.” A amphiphilic oligomer amine derivatizing agent is an amine derivatizing agent including an amphiphilic oligomer and an amine moiety. In an one embodiment, the amphiphilic oligomer is a polyamide, polyester, polyurea, polycarbonate, polyurethane, polyphenylene or heteroarylene polymer. In one embodiment, the amphiphilic oligomer is a polyethylene glycol or polypropylene glycol. In one embodiment, the amphiphilic oligomer is a polyethylene glycol.

In one embodiment, the amine derivatizing agent is a second water soluble polymer amine derivatizing agent. Table 1, entry “j.” A second wafer soluble polymer amine derivatizing agent is an amine derivatizing agent including a second water soluble polymer and an amine moiety. In one embodiment, the second water soluble polymer is a water soluble polymer disclosed herein. In one embodiment, the second water soluble polymer is polyethylene glycol. In one embodiment, the polyethylene glycol is m-dPEG₂₄. See Examples 2 and 12.

In one embodiment, the amine derivatizing agent is a second polypeptide amine derivatizing agent. See Table 1, entry “k.” A second polypeptide amine derivatizing agent is an amine derivatizing agent including a second polypeptide and an amine moiety, wherein the second polypeptide includes a primary or second amine suitable to act as a reactant in the transglutaminase reaction of the methods disclosed herein. In one embodiment, the compound embodiment set forth in Table 1 includes a first polypeptide (i.e., first polypeptide A-P), and a second polypeptide k, wherein the second polypeptide is an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, or an exendin, or analog, derivative or fragment thereof. In one embodiment, a naturally occurring lysine provides the primary amine for the transglutaminase reaction. See Example 10. In one embodiment, an amino acid of the second polypeptide is substituted with lysine, thereby providing a primary amine to serve as a substrate for the transglutaminase. In one embodiment, an amino acid of the second polypeptide is substituted with a homolog of lysine which retains the pendant amine functionality.

In one embodiment, the method provides a compound wherein the first polypeptide further includes a linker. In one embodiment, the method provides a compound wherein the second polypeptide further includes a linker. The terms “linker” and the like, in the context of attachment to a polypeptide refers to a divalent species. In one embodiment, the linker is covalently bonded to a first polypeptide having a valency available for bonding, and to a non-peptidic moiety having a valency available for bonding. In one embodiment, the linker is covalently bonded to a second polypeptide having a valency available for bonding, and to a non-peptidic moiety having a valency available for bonding. In one embodiment, the linker is covalently bonded to a first polypeptide having a valency available for bonding, and to a second polypeptide having a valency available for bonding. It is understood that one valency of a linker disclosed herein may be covalently bonded to a polypeptide or non-peptidic moiety as described herein, and the other valency of the linker may be occupied by a capping moiety, e.g., hydrogen, amine, amide, carboxylate, and the like. Any linker is optional; i.e., any linker may simply be a bond. In one embodiment a linker comprises from 1 to 30 or less amino acids linked by peptide bonds. The amino acids can be selected from the 20 naturally occurring amino acids. Alternatively, non-natural amino acids can be incorporated either by chemical synthesis, post-translational chemical modification or by in vivo incorporation by recombinant expression in a host cell. In one embodiment, one or more amino acids of a linker are glycosylated. In one embodiment, the linker includes a glutamine. In one embodiment, the linker includes a glutamine, which glutamine can react with transglutaminase.

In certain embodiments the amino acids of a linker are selected from glycine, alanine, proline, asparagine, glutamine, lysine, aspartate, serine and glutamate. In one embodiment, the amino acids of a linker are selected from glycine, alanine, proline, asparagine, glutamine, lysine, aspartate, serine and glutamate, wherein glutamine is a required amino acid of the linker. In one embodiment the linker is made up of a majority of amino acids that are sterically unhindered, such as glycine, alanine and/or serine. In one embodiment, the linker includes one or more of acidic linker, a basic linker, and a structural motif. Polyglycines are particularly useful, e.g., (Gly)₃, (GLY)₄, (Gly)₅, as are polyalanines, poly(Gly-Ala), poly(Glyn-Ser), poly(Glyₙ-Glu), poly(Glyₙ-Lys), poly(Glyₙ-Asp), and poly(Glyₙ-Arg) motifs. In one embodiment, the linker includes polyglycine, polyalanines, poly(Gly-Ala), or poly(Gly-Ser). In one embodiment, the linker includes a polyglycine of (Gly)₃, (Gly)₄, or (Gly)₅. Other examples of linkers include (Gly)₃Lys(Gly)₄; (Gly)₃AsnGlySer(Gly)₂; (Gly)₃Cys(Gly)₄; and GlyProAsnGlyGly (SEQ ID NO:57). Combinations of Gly and Ala are particularly useful as are combinations of Gly and Ser. In one embodiment, the linker includes a combination of Gly and Glu. In one embodiment, the linker includes a combination of Gly and Lys. In one embodiment the linker includes a glycine rich peptide, e.g. Gly-Gly-Gly; the sequences [Gly-Ser]ₙ[Gly-Gly-Ser]ₙ, [Gly-Gly-Gly-Ser]ₙ, [Gly-Gly-Gly-Gly-Ser]ₙ, [Gly-Gly]ₙ, [Gly-Gly-Gly]ₙ, [Gly-Gly-Gly-Gly]ₙ and [Gly-Gly-Gly-Gly-Gly]ₙ wherein a is 1, 2, 3, 4, 5, 6, 7, 8, 9, or 10, for example, [Gly-Gly-Gly Ser]₁, [Gly-Gly-Gly-Gly Ser]₁, [Gly-Gly-Gly Ser]₄, or [Gly-Gly-Gly-Gly Ser]₃. The term “glycine rich peptide” refers to a polypeptide having a statistically high glycine content, e.g., a major or preponderance of glycine residues.

In certain embodiments, charged linkers may be used. Such charges linkers may contain a significant number of acidic residues (e.g., Asp, Glu, and the like), or may contain a significant number of basis residues (e.g., Lys, Arg, and the like), such that the linker has a pI lower than 7 or greater than 7, respectively. As understood by the artisan, and all other things being equal, the greater the relative amount of acidic or basic residues in a given linker, the lower or higher, respectively, the pI of the linker will be. Such linkers may impart advantages to the compounds disclosed herein, such as improving solubility and/or stability characteristics of such polypeptides at a particular pH, such as a physiological pH (e.g., between pH 7.2 and pH 7.6, inclusive), or a pH of a pharmaceutical composition comprising such polypeptides.

For example, an “acidic linker” is a linker that has a pI of less than 7; between 6 and 7, inclusive; between 5 and 6, inclusive; between 4 and 5, inclusive; between 3 and 4, inclusive; between 2 and 3, inclusive: or between 1 and 2, inclusive. Similarly, a “basic linker” is a linker that has a pI of greater than 7; between 7 and 8, inclusive; between 8 and 9, inclusive; between 9 and 10, inclusive; between 10 and 11, inclusive; between 11 and 12 inclusive, or between 12 and 13, inclusive. In certain embodiments, an acidic linker will contain a sequence that is selected from the group consisting of [Gly-Glu]ₙ; [Gly-Gly-Glu]ₙ; [Gly-Gly-Gly-Glu]ₙ; [Gly-Gly-Gly-Gly-Glu]ₙ, [Gly-Asp]ₙ; [Gly-Gly-Asp]ₙ; [Gly-Gly-Gly-Asp]ₙ; [Gly-Gly-Gly-Gly-Asp]ₙ where n is 1, 2, 3, 4, 5, 6, 7, 8, 9, 10, or more; for example, [Gly-Gly-Glu]₆.

In certain embodiments, a basic linker will contain a sequence that is selected from the group consisting of [Gly-Lys]ₙ; [Gly-Gly-Lys]ₙ; [Gly-Gly-Gly-Lys]ₙ; [Gly-Gly-Gly-Gly-Lys]ₙ, [Gly-Arg]ₙ; [Gly-Gly-Arg]ₙ[Gly-Gly-Gly-Arg]ₙ; [Gly-Gly-Gly-Gly-Arg]ₙ where n is 1, 2, 3, 4, 5, 6, 7, 8, 9, 10, or more; for example, [Gly-Gly-Lys]₆. In one embodiment, the linker includes a sequence of [Lys]ₙ, wherein n is 1, 2, 3, 4, 5, 6, 7, 8, 9, or 10. In one embodiment, the linker includes the sequence Lys-Lys-Lys-Lys-Lys (SEQ ID NO:58). In one embodiment, the linker has the sequence Lys-Lys-Lys-Lys-Lys (SEQ ID NO:58).

In one embodiment, a linker includes sequence of [Pro]ₙ, wherein n is 1, 2, 3, 4, 5, 6, 7, 8, 9, or 10. In one embodiment, the linker includes the sequence Pro-Pro-Pro-Pro-Pro-Pro (SEQ ID NO:59), in one embodiment, the linker has the sequence Pro-Pro-Pro-Pro-Pro-Pro (SEQ ID NO:59).

Additionally, linkers may be prepared which possess certain structural motifs or characteristics, such as an α helix. For example, such a linker may contain an sequence that is selected from the group consisting of [Glu-Ala-Ala-Ala-Lys]ₙ, where n is 1, 2, 3, 4, 5, 6, 7, 8, 9, 10, or more; for example, [Glu-Ala-Ala-Ala-Lys]₃, [Glu-Ala-Ala-Ala-Lys]₄, or [Glu-Ala-Ala-Ala-Lys]₅. In one embodiments, the linker is selected from the group consisting of Ala-[Glu-Ala-Ala-Ala-Lys]ₙ-Ala, where n is 1, 2, 3, 4, 5, 6,7, 8, 9, 10, or more, preferably n is 3.

Additionally, a non-peptide linker may be employed to serve as the linker moiety of a compound produced by the methods disclosed herein. For example, as understood in the art, an exemplary non-peptide linker such as a PEG linker may be so-employed. See, e.g., WO2000024782. In certain embodiments, such a PEG linker has a molecular weight of 100 Da to 1000 kDa. In certain embodiments, such a PEG linker has a molecular weight of 100 Da to 500 kDa. In certain embodiments, such a PEG linker has a molecular weight of 100 Da to 100 kDa. In certain embodiments, such a PEG linker has a molecular weight of 100 Da to 50 kDa. In certain embodiments, such a PEG linker has a molecular weight of 100 Da to 10 kDa. In certain embodiments, such a PEG linker has a molecular weight of 100 Da to 5 kDa. In certain embodiments, such a PEG linker has a molecular weight of 100 Da to 1 kDa. In certain embodiments, such a PEG linker has a molecular weight of 100 Da to 500 Da.

It is also to be understood that linkers suitable tor use in accordance with the invention may possess one or more of the characteristics and motifs described above. For example, a linker may comprise an acidic linker as well as a structural motif, such as an alpha helix. Similarly, a linker may comprise a basic linker and a structural motif such as an alpha helix. A linker may comprise an acidic linker, a basic linker, and a structural motif, such as an α helix. Additionally, it is also to be understood that compound made in accordance with the methods disclosed herein may possess more than one linker, and each such linker may possess one or more of the characteristics described above.

In one embodiment, the first polypeptide includes an exendin or analog or fragment thereof. In one embodiment, the first polypeptide is exendin-4. In one embodiment, the first polypeptide is exendin-4(1-32). In one embodiment, the first polypeptide is exendin-4(1-31). In one embodiment, the first polypeptide is exendin-4(1-30). In one embodiment, the first polypeptide is exendin-4(1-29). In one embodiment, the first polypeptide is exendin-4(1-28). In one embodiment, the first polypeptide is an analog of exendin-4 having at least 80% sequence identity to exendin-4. In one embodiment, the first polypeptide is an analog of exendin-4 having at least 90% sequence identity to exendin-4. In one embodiment, the first polypeptide is exendin-3.

In one embodiment, any linker disclosed herein further includes a glutamine residue substituted therein in place of a non-glutamine residue. In one embodiment, any linker disclosed herein further includes a glutamine residue appended thereto.

In one embodiment wherein the second polypeptide includes a linker, the linker includes the amine donor.

The linkers described herein are exemplary, and linkers within the scope of this invention may be much longer and may include other residues.

Without wishing to be bound by theory, it is believed that the methods disclosed herein provide a variety of significant advantages for the transglutaminase catalyzed acyl-transfer reactions which afford the covalently bonded compounds disclosed herein. For example, the use of co-solvent in the reaction mixture improves solubility of organic amine. Moreover, the reaction mixture is homogeneous, thereby facilitating enzymatic activity of transglutaminase. Moreover, the currently provided methods afford less side reactions and/or side products, facilitating increases in yield and corresponding decreases in the time and effort required to purify the desired compounds.

### VI. Pharmaceutical Compositions

In another aspect, the present invention provides pharmaceutical compositions comprising compound synthesized by the methods disclosed herein in combination with a pharmaceutically acceptable excipient (e.g., carrier).

The pharmaceutical compositions include optical isomers, diastereomers, or pharmaceutically acceptable salts of the inhibitors disclosed herein. For example, in some embodiments, the pharmaceutical compositions include a compound of the present invention and citrate as a pharmaceutically acceptable salt. The compound included in the pharmaceutical composition may be covalently attached to a carrier moiety, as described above. Alternatively, the compound included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylase or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds disclosed herein can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

A. Formulations

The compounds described herein can be prepared and administered in a wide variety of oral, parenteral, and topical dosage forms. Thus, the compounds described herein can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds can be administered transdermally. It is also envisioned that multiple routes of administration (e.g., intramuscular, oral, transdermal) can be used to administer the compounds. Accordingly, the present description also provides pharmaceutical compositions which include a pharmaceutically acceptable carrier of excipient and one or more compounds described herein.

The compounds described herein can be co-administered to a subject. Co-administration is meant to include simultaneous or sequential administration of the compounds individually or in combination (more than one compound).

The preparations can also be co-administered, when desired, with other active substances (e.g. to reduce metabolic degradation) as known in the art or other therapeutically active agents.

For preparing pharmaceutical compositions from the compounds described herein, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations cart be formulated in solution in aqueous polyethylene glycol solution.

When parenteral application is needed or desired, particularly suitable admixtures for the compounds described herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds described herein can also be incorporated into liposomes or administered via transdermal pumps or patches. Pharmaceutical admixtures suitable for use in the methods described herein include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantify of active component in a unit dose preparation may be varied or adjusted from 0.01 mg to 100 mg, more typically 1.0 mg to 100 mg, most typically 1.0 mg to 50 mg, according to the particular application, the period between each delivery, and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions described herein may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat diabetes, such compositions will contain an amount of active ingredient effective to achieve the desired result (e.g. decreasing fasting blood glucose in a subject). When administered in methods to treat obesity, such compositions will contain an amount of active ingredient effective to achieve the desired result (e.g. decrease the body mass).

The dosage and frequency (single or multiple doses) of compound administered can vary depending upon a variety of factors, including route of administration: size, age, sex, health, body weight, body mass index, and diet of the recipient; nature and extent of symptoms of the disease being treated (e.g., the disease responsive to compounds described herein; fasting blood glucose); presence of other diseases or other health-related problems; kind of concurrent treatment; and complications from any disease or treatment regimen. Other therapeutic regimens or agents can be used in conjunction with the methods and compounds of the invention.

Therapeutically effective amounts for use in humans may be determined from animal models. For example, a dose for humans can be formulated to achieve a concentration that has been found to be effective in animals. The dosage in humans can be adjusted by monitoring one or more physiological parameters, including but not limited to blood sugar and body mass, and adjusting the dosage upwards or downwards, as described above and known in the art.

Dosages may be varied depending upon the requirements of the patient and the compound being employed. The dose administered to a patient, in the context of the present invention, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

However, typical doses may contain from a lower limit of about 1 ug, 5 ug, 10 ug, 50 ug, 100 ug to 150 ug per day to an upper limit of about to 50 ug, to 100 ug, to 150 ug, to 200 ug or even to 5 mg of the pharmaceutical compound. The doses may be delivered in discrete unit doses at the desired interval, e.g. daily or weekly.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p.1, 1975. The exact formulation, route of administration, and dosage can be chosen by the individual physician in view of the patient's condition and the particular method in which the compound is used.

### VII. Examples

### Example 1

### Tramglutaminase Catalyzed PEGylation of Exendin-4

Reaction. One equivalent of exendin-4 amide and five equivalents m-dPEG™ 24 amine were dissolved in buffer, as described below, to a concentration of 1 mg/mL peptide/reaction mixture. The pH was adjusted to 7.5. Aliquots of recombinant bacterial transglutaminase (Zedira, Darmstadt DE) were added, and the reaction was conducted at 37 C and monitored by RP-HPLC. Three solvents system were employed, all of which included 20 mM Tris Buffer, and 5 mM CaCl₂: 1) Control (no co-solvent); 2) 5% propylene glycol; and 3) 5% PEG 300. After 24-hrs the reaction mixture was quenched by bringing the pH to 2.0, and the resulting products were purified and quantified by RP-HPLC. Exendin-4 has a naturally occurring glutamine residue (i.e., ¹³Gln).

Result. The product, ([¹³Q-(dPEG24)]-Exendin-4-NH₂, (MW 4186.66) was observed during reaction as described below. Also observed were a Side Product B (MW 3435.91) and a Side Product A (MW 1840.08), resulting from cleavage of Exendin-4 amide or ([¹³Q]-(dPEG24))-Exendin-4-NH₂ between residues ²¹L and ²²F.

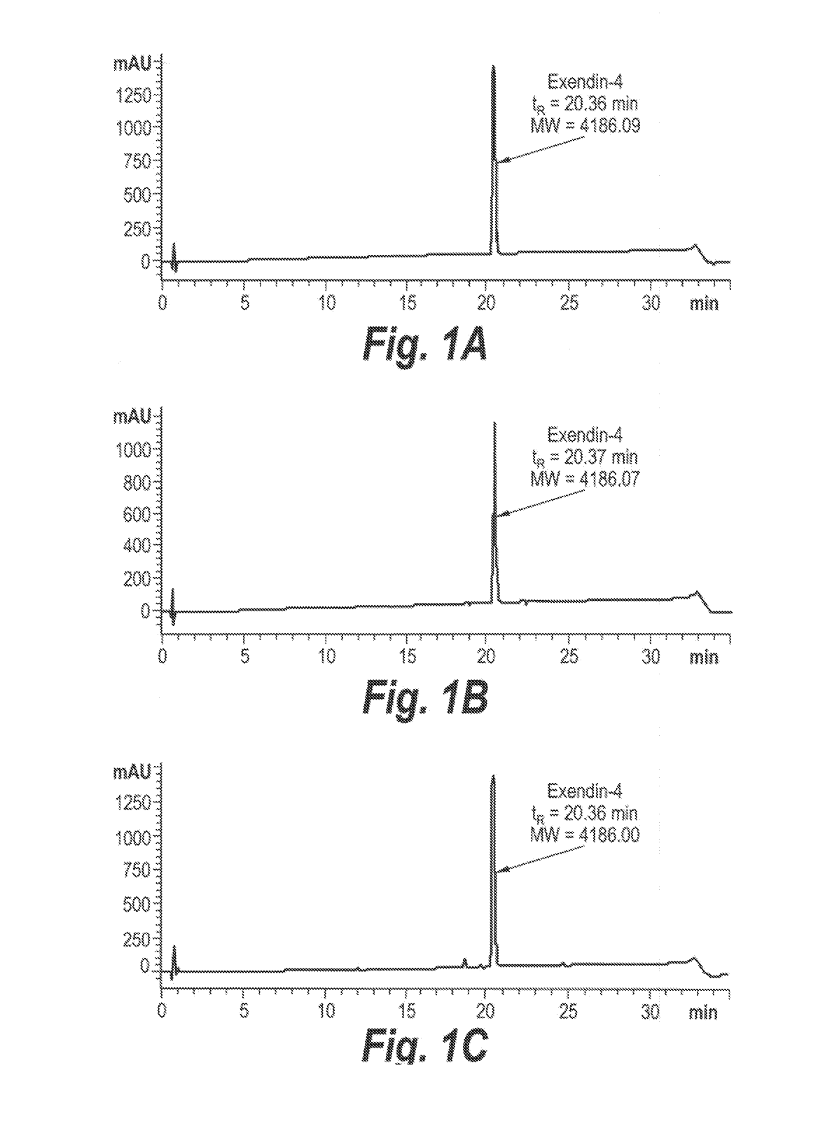

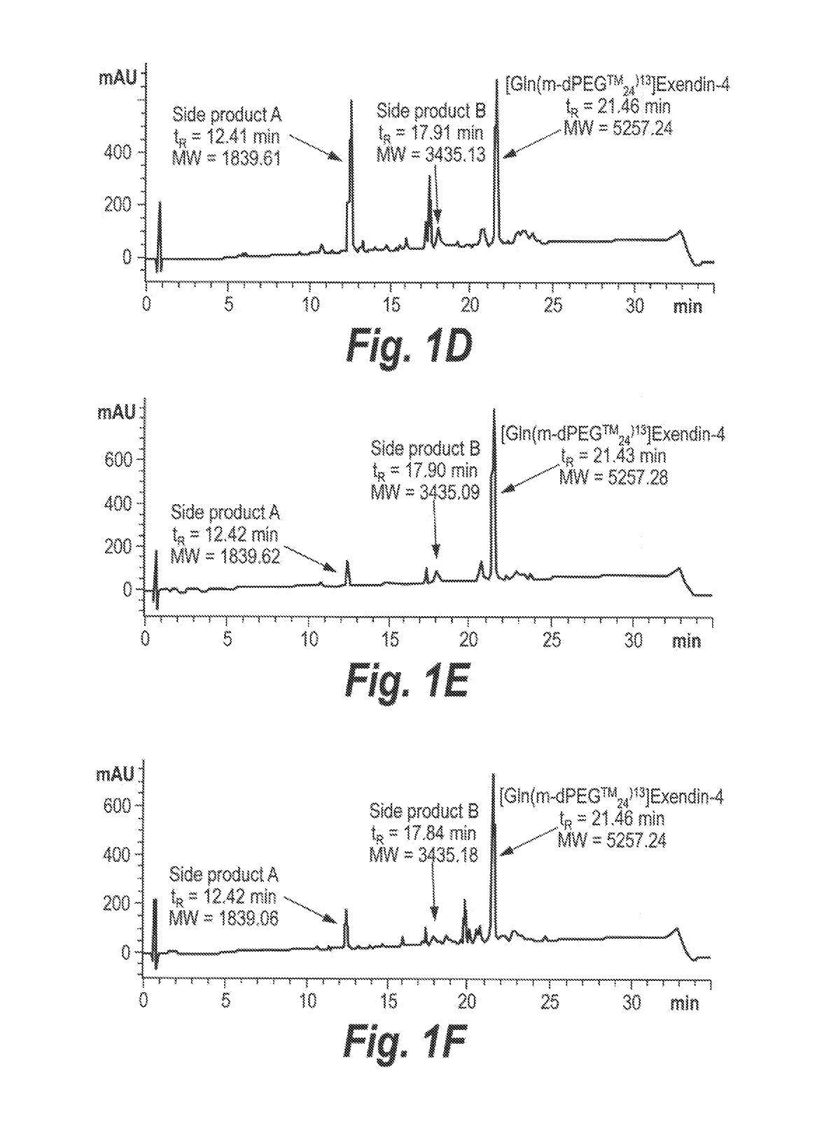

As shown in FIGS. 1A-1C, inclusion of co-solvent resulted in a negligible perturbation of the retention of Exendin-4 in the RP-HPLC system. As shown in FIGS. 1D-1F, after 18-hrs reaction time, virtually all Exendin-4 was consumed in the reaction, affording product ([¹³Q-(dPEG24)]-Exendin-4-NH₂, and side product in varying amounts.

As shown in Table 2 following, the appearance of Side Product A depended upon reaction time and co-solvent content. Specifically, at 2-hrs, Side Product A was observed at 5%, 2%, and 3%, respectively, for control (no co-solvent), 5% propylene glycol, and 5% PEG 300.

TABLE 2
Percent Side Product A in Reaction Mixtures.

| | Side Product A, | Side Product A, |
|---|---|---|
| System | 2-hrs (%) | 18-hrs (%) |
| Control | 5 | 40 |
| 5% Propylene glycol | 2 | 10 |
| 5% PEG 300 | 3 | 15 |

Surprisingly, both 5% propylene glycol and 5% PEG 300 afforded significantly less side product at 18-hrs, compared with control lacking co-solvent.

### Example 2

### Glutamine and dPEG₂₄ Scan of Exendin-4

Reaction. A scan of Exendin-4 amide and analogs thereof was conducted to assess the results of transglutamination to afford pegylated Exendin-4 analog at any of positions 13, 27 and 40.

Results. The GLP-1 binding and functional assays are described above. As shown in Table 3 following,

TABLE 3

| | | GLP-1 | GLP-1 |
|---|---|---|---|
| | | Binding | Function |
| Cmpd | Sequence | IC₅₀ (nM) | ED₅₀ (nM) |
| Ex-4 | Exendin-4 | 0.333 | 0.003 |
| 2-1 | [¹³Q-(dPEG24)]-Exendin-4-NH₂ | 3.189 | 0.009 |
| 2-2 | [des-¹H, ²G(imidazoacetyl), ¹⁴L]Exendin-4-NH₂ | 0.403 | 0.006 |
| 2-3 | [des-¹H, ²G(imidazoacetyl), ¹³Q-(dPEG24), ¹⁴L]Exendin-4-NH₂ | 1.557 | 0.032 |
| 2-4 | [des-¹H, ²G(imidazoacetyl), ¹⁴L, ²⁰Q]Exendin-4-NH₂ | 0.650 | 0.011 |
| 2-5 | [des-¹H, ²G(imidazoacetyl), ¹⁴L, ²⁰Q-(dPEG24)]Exendin-4-NH₂ | 6.071 | 0.1 |
| 2-6 | [des-¹H, ²G(imidazoacetyl), ¹²R, ¹⁴L, ²⁷Q]Exendin-4-NH₂ | 0.962 | 0.014 |
| 2-7 | [des-¹H, ²G(imidazoacetyl), ¹²R, ¹⁴L, ²⁷Q-(dPEG24)]Exendin-4-NH₂ | 2.968 | 0.115 |
| 2-8 | [des-¹H, ²G(imidazoacetyl), ¹⁴L]Exendin-4-⁴⁰Gln-NH₂ | 0.573 | 0.007 |
| 2-9 | [des-¹H, ²G(imidazoacetyl), ¹⁴L]Exendin-4-⁴⁰Gln(dPEG24)-NH₂ | 0.784 | 0.009 |

As shown in Table 3, pegylation with dPEG24 results in a decrease in GLP-1 binding activity, as judged by IC₅₀ results. The decrease in GLP-1 binding activity is about 9,6-fold for Exendin-4 pegylated at ¹³Q. When ¹H is replaced with imidazoacetyl, the decrease in GLP-1 binding activity is observed to be 3.9X, 9.3X, 3.1X, and 1.4X for pegylation at positions 13, 20, 27 and 40, respectively. Similar ranges are observed in the decrease in GLP-1 functional assay activities.

### Example 3

### Time Course of Blood Glucose Change

The compounds disclosed in Example 2 were further assayed for effect on blood glucose. Peptide was injected IP at zero time in 2-hr fasted NIH/Swiss mice. Blood glucose was measured over 4-hrs using a commercially available test kit (OneTouch® Ultra® (LifeScan, Inc. Milpitas, Calif.). The mean pre-treatment blood glucose level was 127 mg/dL.

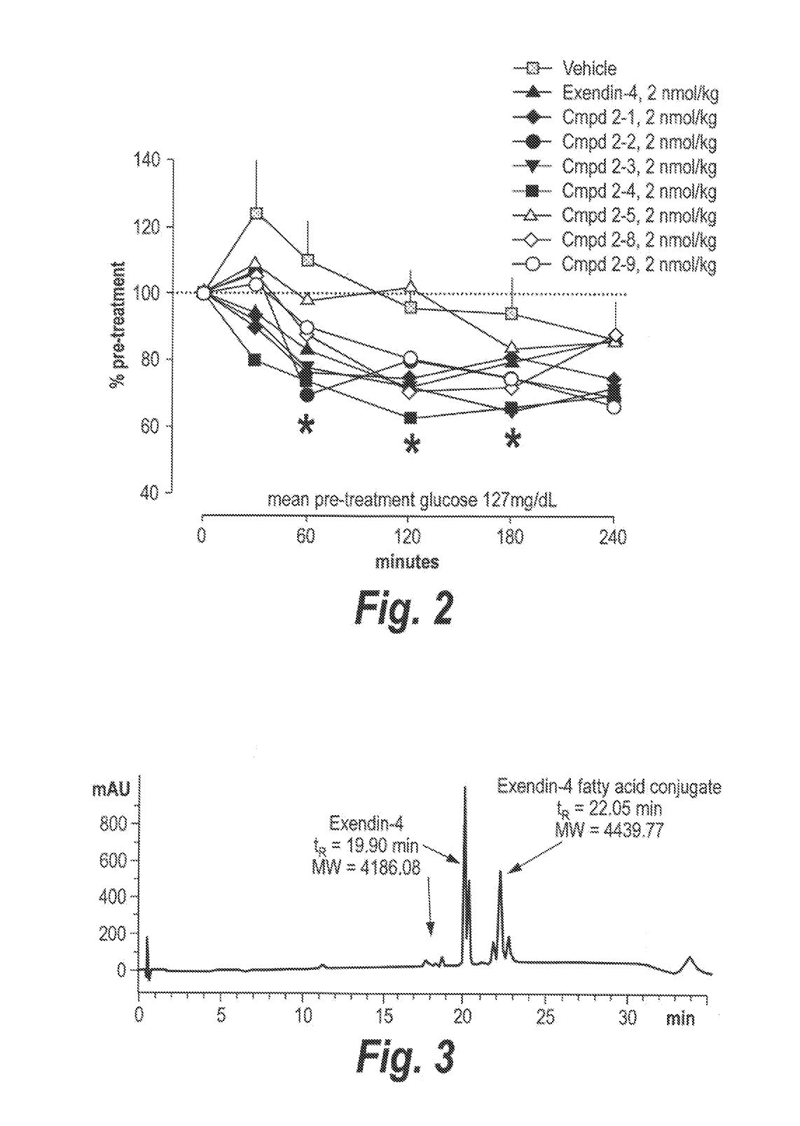

As depicted in FIG. 2, for each of Exendin-4, and Cmpds 2-1, 2-2, 2-3, 2-4, 2-8 and 2-8 blood glucose decreased with respect to vehicle over 2-hrs. At 4-hrs, there was a sustained decrease in blood glucose for Cmpds 2-1, 2-2, 2-3, 2-4, and 2-9.

### Example 4

### Conjugation with Fatty Acid

Using the methods disclosed above, Exendin-4 was covalently linked with the pendant N_{ε}-amine functionality of N_{α}-octan-1-amido-lysine, shown following, at the naturally occurring ¹³Q residue of Exendin-4. As depicted in FIG. 3, the desired [¹³Q(N_{α}-octan-1-amido lysine)]Exendin-4 product was observed.

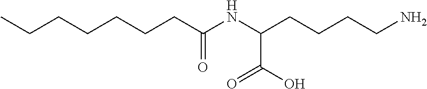 [SMILES: CCCCCCCC(=O)NC(CCCCN)C(=O)O]

### Example 5

### Conjugation with Cholic Acid

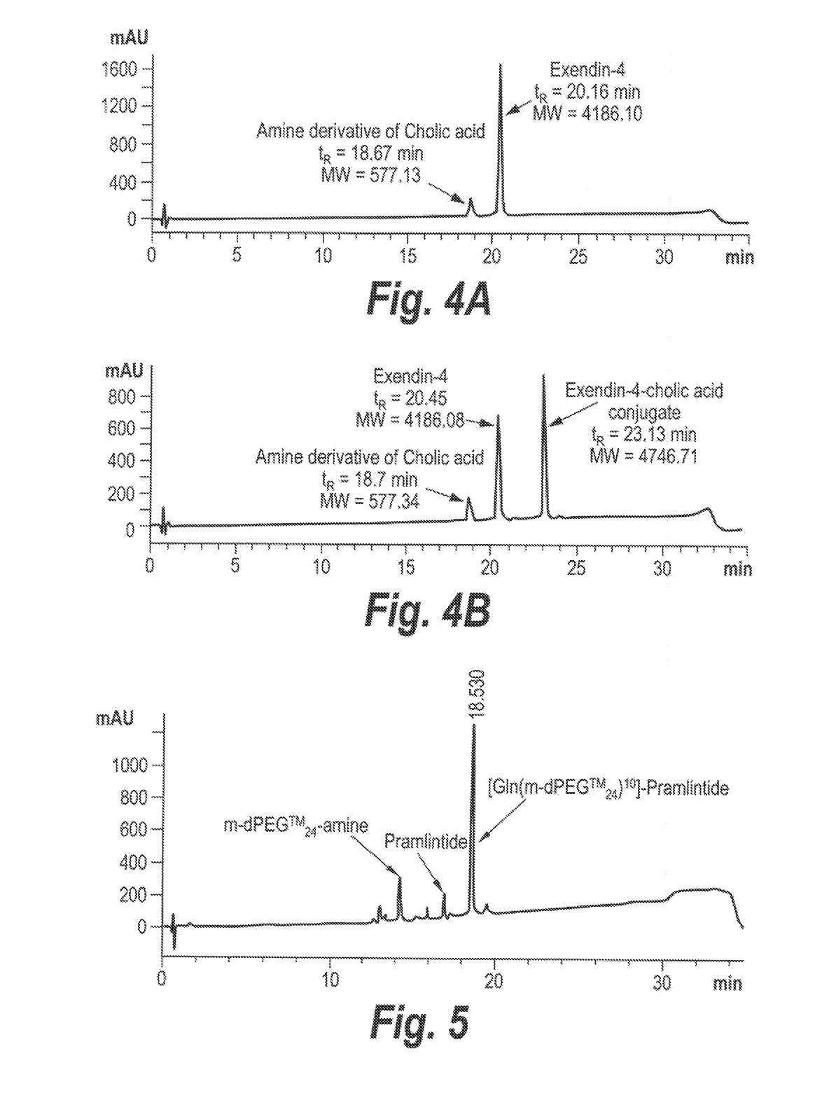

Using the methods disclosed above, Exendin-4 was covalently linked with a cholic acid derivative at the naturally occurring ¹³Q residue of Exendin-4. The cholic acid derivative included a glycyllysyl dipeptide in amide linkage at the cholic acid carboxylate functionality, shown following. The pendant N_{ε}-amine functionality of the terminal lysine of the cholic acid derivative served as a substrate for the transglutaminase reaction. As depicted in FIG. 4A, Exendin-4 and the cholic acid dipeptide are observed in the initial (zero time) RP-HPLC chromatogram. As depicted in FIG. 4B, the desired. [¹³Q(cholic acid-glycyllysyl amide)]Exendin-4 product was observed at the 8-hr time point.

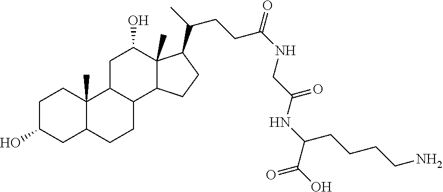 [SMILES: CC(CCC(=O)NCC(=O)NC(CCCCN)C(=O)O)[C@H]1CCC2C3CCC4C[C@H](O)CC[C@]4(C)C3C[C@H](O)[C@@]21C]

### Example 6

### Conjugation with Biotin

Using the methods disclosed above, Exendin-4 and an analog were covalently linked with biotin cadaverine amine, shown following.

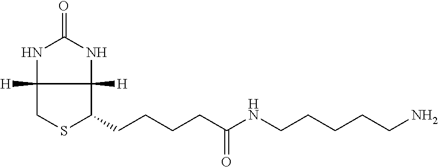 [SMILES: NCCCCCNC(=O)CCCC[C@@H]1SC[C@@H]2NC(=O)N[C@@H]21]

Exendin-4. Reaction of the transglutaminase with biotin cadaverine amide, via the pendant amine, at position ¹³Q of Exendin-4 afforded a species (MW 4497.15) having IC₅₀ 0.338 nM and EC₅₀ of 0.004 nM in the GLP-1 binding and functional assays described above.

[des-¹H, ²G(imidazoacetyl), ¹³N, ¹⁴L]-Exendin-4-⁴⁰Q. The Exendin-4 analog lacking an N-terminal amine functionality and having substitutions Q13N and M14L and a glutamine residue appended at the C-terminal was subjected to the transglutaminase reaction described above with biotin cadaverine amide. The resulting covalently attached biotin cadaverine amide adduct (MW 4564.46) attached at ⁴⁰Q had an IC₅₀ of 0.604 nM and an EC₅₀ of 0.004 nM in the GLP-1 binding and functional assays, respectively.

### Example 7

### Conjugation with Vitamin B12

Using the methods disclosed above, [des-¹H, ²G(imidazoacetyl), ¹³N, ¹⁴L]-Exendin-4-⁴⁰Q was covalently linked with the Vitamin B12 derivative shown following (MW 5734.08). The resulting compound had an IC₅₀ of 0.423 nM and an EC₅₀ of 0.033 nM in the GLP-1 binding and functional assays, respectively.

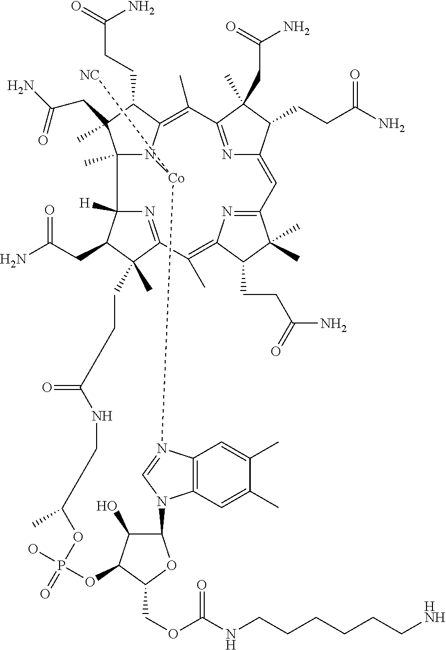

### Example 8

### Conjugation with Dansyl Cadaverine

Exendin-4 was subjected to the transglutaminase reaction described above in the presence of dansyl cadaverine, which resulted in covalent attachment of the fluorescent label at ¹³Q of Exendin-4. The resulting compound had an EC₅₀ of 0.003 nM is the GLP-1 functional assay.

### Example 9

### Conjugation with CCK-8

[¹²R, ¹³N, ¹⁴L]-Exendin-4-GGGQ was subjected to the transglutaminase reaction described above in the presence of a CCK-8 analog having an N-terminal lysine: KDF(sulfate)MGWMDF-NH₂ to afford the Exendin analog-CCK-8 covalently bonded compound: MS (MALDI) MW=5821.94.

### Example 10

### Conjugation with [¹⁰N]-Pramlintide

[¹²R, ¹³N, ¹⁴L]-Exendin-4-GGGQ was reacted with [¹⁰N]-pramlintide under the reaction conditions described above to afford the dual peptide exendin analog-pramlintide analog compound. MS (MALDI): MW=8426.53.

### Example 11

### Conjugation with Zot Derivative

[¹²R, ¹³N, ¹⁴L]-Exendin-4-GGGQ was reacted with FXIGRLK-amide, where X is allylglycine, under the reaction conditions described above to afford the covalently bonded compound: MS (MALDI): MW-5320.33.

### Example 12

### Conjugation of Pramlintide with dPEG24

Pramlintide, having a glutamine at position 10, was reacted with m-dPEG24 amine as described above to afford the pegylated compound. As depicted in FIG. 5, after 3-hrs of reaction, the resulting compound is well separated from reagents m-dPEG24-amine and pramlintide. The resulting compound has an observed IC₅₀ of 80 nM in the GLP-1 binding assay.

### Example 13

### Conjugation of (O-succinoyl) Vitamin B12 as Conjugation Substrate on Exendin-4.

Site specific chemical conjugation of Exendin-4 having two side chain lysines at positions 12 and 27 is practically impossible and requires mutation of one of the lysines with amino acids such as Arginine (scheme. 1).

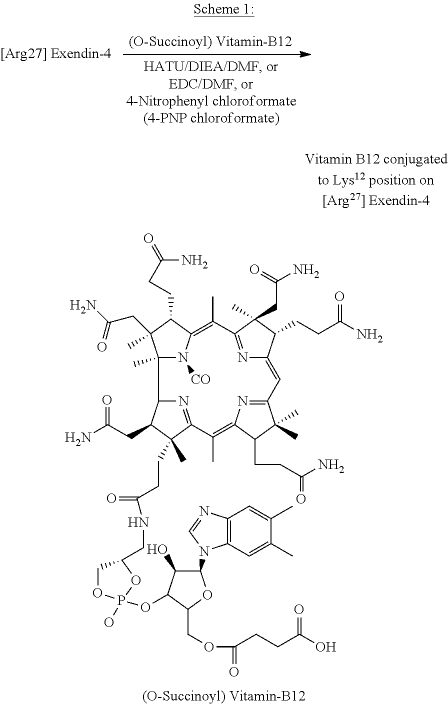

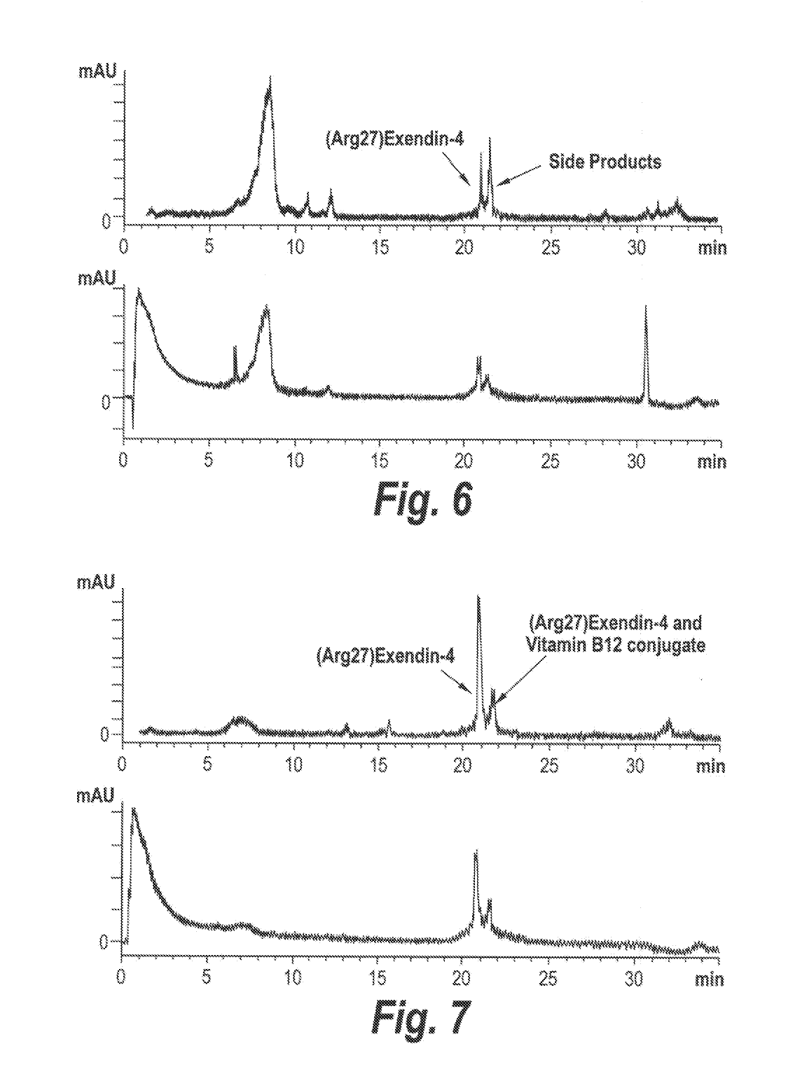

A chemical conjugation using HATU/DIEA in DMF as a coupling agent during the solid phase peptide synthesis did not yield any desired product and many conjugation substrates are not compatible under those harsh solid support cleavage conditions. A solution phase chemical conjugation reaction using either EDC (FIG. 6) or 4-PNP as an activating reagent produced about 10% product (FIG. 7). In contrast, enzymatic conjugation on Exendin-4 doesn't require any amino acid mutations and the reaction products are very clean.

### VII. Embodiments

### Embodiment 1

A method of covalently attaching an amine derivatizing agent to a glutamine of a first polypeptide, said method comprising: a) adding an amine derivatizing agent, a transglutaminase, a first polypeptide comprising a glutamine, and a co-solvent to a reaction mixture; and b) allowing said amine derivatizing agent to react with said glutamine in said reaction mixture to form an amide bond; thereby covalently attaching said amine derivatizing agent to said glutamine of said first polypeptide.

### Embodiment 2

The method according to embodiment 1, wherein the extent of covalently attaching said amine derivatizing agent to said glutamine of said first polypeptide is at least 50%.

### Embodiment 3

The method, according to embodiment 2, wherein the extent of covalently attaching said amine derivatizing agent to said glutamine of said first polypeptide is at least 80%.

### Embodiment 4

The method according to embodiment 1, wherein said co-solvent is a first water-soluble polymer.

### Embodiment 5

The method according to embodiment 1, wherein said reaction mixture comprises at least 5% (w/w) of said co-solvent.

### Embodiment 6

The method according to embodiment 5, wherein said co-solvent is a glycol.

### Embodiment 7

The method according to embodiment 6, wherein said glycol is propylene glycol or polyethylene glycol.

### Embodiment 8

The method according to embodiment 1, wherein a lower concentration of side reaction products obtains relative to the corresponding method wherein said co solvent is absent from said reaction mixture.

### Embodiment 9

The method according to embodiment 8, wherein said lower concentration of side reaction products is 25% or less relative to the corresponding method wherein, said co solvent is absent from said reaction mixture.

### Embodiment 10

The method according to any one of embodiments 1 to 8, wherein said first polypeptide comprises a peptide hormone, or analog or fragment thereof.

### Embodiment 11

The method according to embodiment 10, wherein said first polypeptide covalently attached to said amine derivatizing agent exhibits at least one hormonal activity.

### Embodiment 12

The method according to embodiment 10, wherein said first polypeptide comprises an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog or fragment thereof.

### Embodiment 13

The method according to embodiment 12, wherein said first polypeptide further comprises a linker.

### Embodiment 14

The method, according to embodiment 13, wherein said linker comprises said glutamine.

### Embodiment 15

The method according to embodiment 12, wherein said glutamine is a naturally occurring glutamine.

### Embodiment 16

The method according to embodiment 12, wherein said first polypeptide comprises an exendin, or analog or fragment thereof.

### Embodiment 17

The method according to embodiment 16, wherein said exendin analog has at least 80% sequence identity to exendin-4.

### Embodiment 18

The method according to embodiment 17, wherein said exendin analog has at least 90% sequence identity to exendin-4.

### Embodiment 19

The method according to embodiment 16, wherein said exendin is exendin-3 or exendin-4.

### Embodiment 20

The method according to embodiment 19, wherein, said exendin is exendin-4.

### Embodiment 21

The method according to embodiment 12, wherein said amine derivatizing agent comprises an imaging label, a fluorescent label, a fatty acid, a bile acid, a glycan, a nasal delivery enhancing compound, biotin, cobalamine or derivative thereof, an amphiphilic oligomer, a second water soluble polymer, or a second polypeptide.

### Embodiment 22

The method according to embodiment 21, wherein said amine derivatizing agent comprises an imaging label.

### Embodiment 23

The method according, to embodiment 21, wherein said amine derivatizing agent comprises a fluorescent label.

### Embodiment 24

The method according to embodiment 21, wherein said amine derivatizing agent comprises a fatty acid.

### Embodiment 25

The method according to embodiment 21, wherein said amine derivatizing agent comprises a bile acid.

### Embodiment 26.

The method according to embodiment 25, wherein said bile acid is cholic acid.

### Embodiment 27

The method according to embodiment 21, wherein said amine derivatizing agent comprises biotin.

### Embodiment 28

The method according to embodiment 21, wherein said amine derivatizing agent comprises said second water soluble polymer.

### Embodiment 29

The method according to embodiment 28, wherein said second water soluble polymer is polyethylene glycol.

### Embodiment: 30

The method according to embodiment 21, wherein said second polypeptide is an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog or fragment thereof.

### Embodiment 31

The method according to embodiment 21, wherein said second polypeptide comprises a linker.

### Embodiment 32

The method according to embodiment 31, wherein said linker comprises said amine donor.

1. A method of covalently attaching an amine derivatizing agent to a glutamine of a first polypeptide, said method comprising:
a) adding an amine derivatizing agent, a transglutaminase, a first polypeptide comprising a glutamine, and a co-solvent to a reaction mixture; and
b) allowing said amine derivatizing agent to react with said glutamine in said reaction mixture to form an amide bond;
thereby covalently attaching said amine derivatizing agent to said glutamine of said first polypeptide.

2. The method according to claim 1, wherein the extent of covalently attaching said amine derivatizing agent to said glutamine of said first polypeptide is at least 50%.

3. (canceled)

4. The method according to claim 1, wherein said co-solvent is a first water-soluble polymer.

5. The method according to claim 1, wherein said reaction mixture comprises at least 5% (w/w) of said co-solvent.

6. The method according to claim 5, wherein said co-solvent is a glycol.

7. The method according to claim 6, wherein said glycol is propylene glycol or polyethylene glycol.

8. The method according to claim 1, wherein a lower concentration of side reaction products obtains relative to the corresponding method wherein said co solvent is absent from said reaction mixture.

9. The method according to claim 8, wherein said lower concentration of side reaction products is 25% or less relative to the corresponding method wherein said co solvent is absent from said reaction mixture.

10. The method according to claim 1, wherein said first polypeptide comprises a peptide hormone, or analog or fragment thereof.

11. (canceled)

12. The method according to claim 10, wherein said first polypeptide comprises an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog or fragment thereof.

13. The method according to claim 12, wherein said first polypeptide further comprises a linker.

14-15. (canceled)

16. The method according to claim 12, wherein said first polypeptide comprises an exendin, or analog or fragment thereof.

17. The method according to claim 16, wherein said exendin analog has at least 80% sequence identity to exendin-4.

18. (canceled)

19. The method according to claim 16, wherein said exendin is exendin-3 or exendin-4.

20. The method according to claim 19, wherein said exendin is exendin-4.

21. The method according to claim 12, wherein said amine derivatizing agent comprises an imaging label, a fluorescent label, a fatty acid, a bile acid, a glycan, a nasal delivery enhancing compound, biotin, cobalamine or derivative thereof, an amphiphilic oligomer, a second water soluble polymer, or a second polypeptide.

22-25. (canceled)

26. The method according to claim 21, wherein said bile acid is cholic acid.

27-28. (canceled)

29. The method according to claim 21, wherein said second water soluble polymer is polyethylene glycol.

30. The method according to claim 21, wherein said second polypeptide is an amylin, pramlintide, adrenomedullin (ADM), calcitonin (CT), calcitonin gene related peptide (CGRP), intermedin, a cholecystokinin (CCK), a leptin, peptide YY (PYY), glucagon-like peptide-1 (GLP-1), glucagon-like peptide 2 (GLP-2), oxyntomodulin (OXM), a natriuretic peptide, a urocortin, a neuromedin family peptide, an exendin, or analog or fragment thereof.

31. The method according to claim 21, wherein said second polypeptide comprises a linker.

32. (canceled)
